SEQUENCE LISTING (3 sequences)

SEQ ID NO: 1 - protein, 8 aa, Artificial Sequence
  mammal
NQEQVSPL

SEQ ID NO: 2 - protein, 14 aa, Artificial Sequence
  mammal
NQEQVSPLRGDSPG

SEQ ID NO: 3 - protein, 7 aa, Artificial Sequence
  mammal
FKGGERC

(19) United States
(12) Patent Application Publication
(10) Pub. No.: US 2015/0104427 A1
(43) Pub. Date: Apr. 16, 2015

(54) ENZYME-ASSISTED SPATIAL DECORATION OF BIOMATERIALS

(71) Applicant: THE REGENTS OF THE UNIVERSITY OF CALIFORNIA, Oakland, CA (US)

(72) Inventor: Tatiana Segura, Los Angeles, CA (US)

(73) Assignee: The Regents of the University of California, Oakland, CA (US)

(21) Appl. No.: 14/357,545

(22) PCT Filed: Nov. 9, 2012
(86) PCT No.: PCT/US2012/064475

§ 371 Date: May 9, 2014

Related U.S. Application Data
(60) Provisional application No. 61/629,021, filed on Nov. 10, 2011.

(51) Int. Cl. A61K 38/45

(52) U.S. Cl. CPC A61K 38/45; C12Y 203/02013
USPC 424/93.7; 424/94.5

(57) ABSTRACT

A method of producing a hydrogel compromising a spatially-controlled, three-dimensional distribution of one or more bioactive signals is provided. The method compromises illuminating the hydrogel, wherein the hydrogel compromises a polymer bound to a peptide comprising a photolabile protected amino acid, wherein at least one portion of the hydrogel is illuminated to deprotect the protected amino acid, thereby converting the protected amino acid to a deprotected amino acid. Preferably, the deprotected amino acid is a substrate for an enzyme in at least one portion of the hydrogel. The method further comprises the step of contacting the hydrogel with the enzyme and a peptide comprising a bioactive signal, wherein the enzyme can form a bond between the substrate and the peptide comprising the bioactive signal, thereby producing a hydrogel compromising a plurality of bioactive signals occupying three dimensions of the hydrogel within at least one portion of the hydrogel subjected to illumination.

### CROSS-REFERENCE TO RELATED APPLICATIONS

This application claims priority to U.S. Provisional Application No. 61/629,021 filed on Nov. 10, 2011, the entire contents of which are hereby incorporated by reference in its entirety.

### STATEMENT AS TO RIGHTS TO INVENTORS MADE UNDER FEDERALLY SPONSORED RESEARCH AND DEVELOPMENT

This invention was made with Government support of Grant No. EB009516, awarded by the National Institutes of Health. The Government has certain rights to this invention.

### FIELD OF INVENTION

The present invention relates to the area of tissue engineering. In particular, the invention relates to methods and compositions for specifically immobilizing bioactive signals with spatial control.

### BACKGROUND OF THE INVENTION

Our understanding of how the spatial and temporal regulation of signals regulate stem cell differentiation and tissue morphogenesis in vertebrate animals has been principally obtained from the studies of lower organisms such as zebrafish embryos [(see, e.g., Aman A, Piotrowski T. Cell migration during morphogenesis. Dev Biol May 1; 341(1):20-33)]. Zebrafish embryos are ideal for developmental biology studies and to study tissue morphogenesis because they are optically clear allowing real-time live imaging of morphogenesis. However, these organisms are still remarkably complex with a myriad of signals acting in concert to result in morphogenesis. Although elegant genetic studies may be designed with zebrafish to study the importance and mechanism of specific genes and pathways, the complexity of the system makes it difficult to determine the minimum signals required to achieve morphogenesis or stem cell differentiation and to identify the mechanism of action. Most mechanistic studies and detailed molecular biology studies are performed with cells cultured in two-dimensions. Although one can perform controlled studies, culturing of cells in two-dimensions does not represent what happens to cells in vivo. [(see, e.g., Fraley S I, Feng Y, Krishnamurthy R, Kim D H, Celedon A, Longmore G D, et al. A distinctive role for focal adhesion proteins in three-dimensional cell motility. Nat Cell Biol June; 12(6):598-604) and Gu Z, Tang Y. Enzyme-assisted photolithography for spatial functionalization of hydrogels. Lab Chip August 7; 10(15):1946-1951)].

The use of UV labile groups (not peptides) to immobilize or degrade bioactive signals with spatial control has been previously explored by others [(see, e.g., 24. Kloxin A M, Kasko A M, Salinas C N, Anseth K S. Photodegradable hydrogels for dynamic tuning of physical and chemical properties. Science 2009 Apr. 3; 324(5923):59-63 and Luo Y, Shoichet M S. A photolabile hydrogel for guided three-dimensional cell growth and migration. Nat Mater 2004 April; 3(4):249-253, Burdick, J. A.; Khademhosseini, A.; Langer, R. Langmuir 2004, 20, 5153; Kloxin, A. M.; Kasko, A. M.; Salinas, C. N.; Anseth, K. S. Science 2009, 324, 59; Wong, D. Y.; Griffin, D. R.; Reed, J.; Kasko, A. M. Macromolecules 2010, 43, 2824)]. However, these approaches are employed in 2-D and/or use unspecific chemistries to immobilize the bioactive signals (e.g. amine or thiol chemistry). Therefore, it is important to design and synthesize in vitro cell culture platforms that can be used to bridge the gap between the elegant studies performed in zebrafish embryos and tissue culture plates. Such in vitro cell culture platforms will provide more “realistic” systems to identify the key mechanisms by which stem cells are differentiated, multicellular structures are organized and tissue morphogenesis as a whole occurs. Further, once the environments and signals are identified that result in stem cell differentiation and morphogenesis, these environments may be used to promote tissue formation and regeneration in vivo through the induction of endogenous or transplanted stem cells at the injured or diseased site. Advances in developmental, cell and molecular biology are beginning to map out the spatial and temporal regulation of bioactive and physical signals required for the orchestration of tissue formation. This spatial regulation of bioactive signals is most dramatic during embryogenesis where a whole organism is created from just one cell. However, the fine tuned orchestration of tissue formation also occurs in adults during wound healing and homeostasis. In many instances, one signal and one static environment are insufficient for tissue formation or homeostasis. Thus, the ability to probe human stem cell fate in vitro and determine what are the minimum required signals to result in tissue formation or a desired stem cell fate is limited by our current inability to create cellular microenvironments with complex and dynamic patterns of signals and cellular microenvironments where morphogenesis/differentiation can be visualized in real-time. Accordingly, there is a need for a more “realistic” in vitro culturing platform that can recapitulate the in vivo complexity and allow for live imaging.

### SUMMARY OF THE PREFERRED EMBODIMENTS

A method of producing a hydrogel is provided. The method compromises a spatially-controlled, three-dimensional distribution of one or more bioactive signals, comprising illuminating the hydrogel, wherein the hydrogel compromises a polymer bound to a peptide comprising a photolabile protected amino acid, wherein at least one portion of the hydrogel is illuminated to deprotect the protected amino acid, thereby converting the protected amino acid to a deprotected amino acid, wherein the deprotected amino acid is a substrate for an enzyme in at least one portion of the hydrogel; and contacting the hydrogel with the enzyme and a bioactive signal, wherein the enzyme can form a bond between the substrate and the peptide comprising the bioactive signal, thereby producing a hydrogel compromising a plurality of bioactive signals occupying three dimensions of the hydrogel within at least one portion of the hydrogel subjected to illumination. Preferably, the bond is a covalent bond. Preferably, the polymer comprises hyaluronic acid and/or poly(ethylene glycol). Preferably, the protected amino acid is a caged amino acid selected from the group consisting of lysine (K), aspartic acid (D), glutamic acid (E), arginine (R), serine (S), tyrosine (Y), and cysteine (C). More preferably, the protected amino acid compromises a caged lysine (K). Preferably, the enzyme compromises Factor XIIIa. Preferably, the bioactive signal comprises an amino acid glutamine (Q) linked to an amino acid motif RGD. Preferably, the caged amino acid comprises an ortho-nitrobenzyl photoactive chemical moiety. Preferably, the enzyme Factor XIIIa catalyzes a transamination reaction between the deprotected amino acid and the bioactive signal, thereby immobilizing the bioactive signal to the hydrogel. Preferably, the method compromises the step of seeding the hydrogel with cells. In a preferred embodiment, a hydrogel formed by foregoing method is provided. In a preferred embodiment, a method of controlling cellular migration and/or introducing tunnels in hydrogels, comprising using the hydrogel formed by the foregoing methods.

In a preferred embodiment, a method of producing a hydrogel is provided. The method compromises illuminating the hydrogel. Preferably, the hydrogel compromises a polymer bound to a photolabile protected peptide. Preferably, one or more portions of the hydrogel is illuminated to deprotect the photolabile protected peptide. Accordingly, the photolabile protected peptide is converted to a deprotected peptide. The deprotected peptide is a substrate for an enzyme in one or more portions of the hydrogel; thereby degrading the peptide within one or more portions of the hydrogel subjected to illumination. Preferably, the peptide is a protease degradable peptide and/or compromises at least one protease cleavage site. More preferably, the peptide is a MMP degradable peptide. Preferably, the enzyme is a protease. More preferably, the enzyme is MMP protease. Preferably, the peptide is a peptide degradable by trypsin or plasmin Preferably, the enzyme is trypsin or plasmin Preferably, the method compromises the step of seeding the hydrogel with cells.

In a preferred embodiment, the peptide and/or the bioactive signal compromises a protease cleavage site. Preferably, cleavage at said site releases a bioactive signal.

### DESCRIPTION OF THE DRAWINGS

FIG. 1. Spacially Patterning Bioactive Signal Incorporation.

FIG. 2. 2D Patterning of RGD Bioactive Signal.

FIG. 3. Spatially Patterning Bioactive Signal Incorporation. A. Scheme to use FXIIIa caged peptide to achieve spacial signal immobilization. B Immobilization of FITC-labeled Q-RGD to PEG-VS/K/MMP hydrogels using FXIIIa. C. Synthesis of caged K peptide. D. Deprotection of photocaged K* peptide under exposure to 365 nm light (I=20 mW/cm2) [Step 1]. E. Enzymatic attachment of FITC-labeled Q-RGD into PEG-VS/K/MMP hydrogels using FXIIIa [Step 2].

FIGS. 4 and 4B. Synthetic Scheme for K Star Peptide.

FIG. 5. A. Scheme to use a FXIIIa caged peptide to achieve bioactive signal immobilization. B Immobilization of Q-RGD to HA-AC/K/MMP hydrogels using FXIIIa and culture of hMSCs. C. Synthesis of caged lysine (K*) and caged K peptide (K* peptide). D. Spacially controlled immobilization of FITC-Q-RGD. E. Spacially controlling bioactive signal incorporation. F. Two bioactive signals plus flow.

FIG. 6. Spacially Controlling Cell Migration.

FIG. 7. Spacially Controlling Cell Migration.

FIG. 8. Synthesis of K Star Sequence. The synthesis of the photoprotected “K” Star peptide involves th reductive amination of the lysine of the unprotected “K” peptide using an ortho-nitrobenzyl photoactive chemical moiety. The synthesis is a simple one-step modification of the “K” peptide, which allows for ease of synthesis and a single purification step.

FIG. 9. Confirmation of K Star Synthesis. The liquid Chromotography-Mass Spectrometry (LCMS) readout shows the presence of only the intended photoprotected peptide, which has a molecular weight of 1104 Da. This molecular weight corresponds to an expected m/z value of 553, as seen in the inset graph.

FIG. 10. Characterization of K Star Photodegradation. The degradation behavior of the photocaged K Star peptide upon exposure to 365 nm light was captured as a function of time by using a TNBSA assay to quantify the concentration of free amines after different periods of exposure. The equation represents a quantitative estimate of the concentration of unprotected peptide for a given intensity (I₃₆₅) and time of exposure (t).

FIG. 11. Enzymatic Activity Within Hydrogels. The immobilization of a peptide, Q-RGD (derived from natural cell matrix protein), was followed by fluorescently tagging the peptide before immobilization and measuring the concentration still in solution following immobilization. The K Star test was performed through exposure for 10 minutes at 4 mW/cm2 (corresponding to 47.8% deprotection by the equation in FIG. 10). The predicted value (213 uM) is based on the calculated deprotection and compares well to the actual value (203 uM).

FIG. 12. Biomolecular Patterning With Hydrogels. Hydrogels were exposed to 365 nm light through photomasks and then placed in the presence of thrombin (to activate encapsulated Factor XIIII) and Q-RGD to allow for biomolecule immobilization. The immobilized peptide was able to be imaged by fluorescently tagging the peptide before immobilization, followed by leaching of the unattached peptide and imaging on a fluorescent microscope.

FIG. 13. The K peptide can also be synthesized by first modifying a lysine amino acid, followed by solid phase peptide synthesis. A. Scheme to use a FXIIIa caged peptide to achieve bioactive signal immobilization. B Immobilization of Q-RGD to HA-AC/K/MMP hydrogels using FXIIIa and culture of hMSCs. C. Synthesis of caged lysine (K*) and caged peptide (K* peptide). D. Spatially controlled immobilization of FITC-Q-RGD.

FIG. 14. Current “homogeneous” hydrogel scaffolds and proposed heterogeneous and dynamic hydrogel scaffolds. Not shown is the ability to add soluble signal concentration gradients.

FIG. 15. Strategy to immobilize bioactive signals with spacial control into hydrogel scaffolds using FXIIIa chemistry. A. Scheme and peptide sequences. B. Shows FXIIIa may be added post hydrogel formation. C. Shows K* and K* peptide synthesis. D. Shows that RGD can be immobilized with spatial control inside hydrogel scaffolds.

FIG. 16. Multiple bioactive signals can be incorporated to K* peptide containing hydrogels through sequential deprotection/binding steps.

FIG. 17. Stop regions in the cartoon represent no MMP-2 activity. However, the entire hydrogel has an MMP-1 when tunnels are not to be formed.

FIG. 18. Cellular filtration in HA hydrogel scaffolds. H&E stain after 7 days of implantation.

FIG. 19. Schematic of the microfluidic device used in a preferred embodiment of the present invention.

FIG. 20. Schematic of a preferred embodiment of the present invention showing bioactive signal incorporation and in vitro mMSC experiments.

FIG. 21. Schematic of a preferred embodiment of the present invention showing: Fn fragment engineering. Fn immobilization in 2D and hES-MEC differentiation. Optimization of Q-FN immobilization in 3D and hES-MEC cell culture in 3D. Morphogenesis in spacially engineered scaffolds and in vivo angiogenesis.

FIG. 22. Quantitative analysis via SPR integrin (α3β1) binding to engineered Fn fragments. A. Immobilization of purified, functional soluble integrin; B. SPR sensorgrams of Fn fragment binding kinetics. C.-F. Epithelial cell attachment to specific engineered Fn fragments (variant 1: C.E; Variant 2: D, F) in the presence of function blocking antibodies (C, D) or soluble adhesive peptides (E, F).

FIG. 23. Integrin clustering on engineered Fn fragments (variant 1: A, C, E, G; variant 2: B, D, F, H). A-B) Actin, C-D) intergrin α3, E-F) integrin αV, G-H) integrin α5.

FIG. 24. Immobilization of Q-RGD to HA-K/MMP using FXIIIa. FXIIIa is required for Q-RGD binding (A). Encapsulated means FXIIIa is added during hydrogel formation and added after means that FXIIIa is added after hydrogel formation. B. Characterization of the synthesis of caged lysine and caged K* peptide. (C) Spatial immobilization of Q-RGD to HA-AC/K*/MMP after deprotection of K* using a mask with lines. Q-RGD is only grafted where light was allowed to pass through.

FIG. 25. Analysis of epithelial precursor cell EMT on engineered Fn fragments. White plots represent epithelial differentiation while gray plots represent mesenchymal differentiation. Fragments that support α5 and α3 engagement facilitate epithelial differentiation (left bars on graphs and panels A & E; E-cadherin and αSMA respectively), whereas fragments that primarily engage αv integrin drive EMT and mesenchymal differentiation (right columns on graphs and panels B&F; E-cadherin and αSMA, respectively). Cells treated with TGFβ display EMT regardless of the adhesion ligands (panels C, D, and G, H).

FIG. 26. Schematic of experimental conditions in a preferred embodiment of the invention.

FIG. 27. HA-AC/MMP hydrogel mechanical properties show a wide range of storage modulus G′ for different conditions (A), mMSC speading inside HA-AC/MMP shows that high RGD concentration leads to more spread cells; (B) and mMSC proliferation for cells inside HA-AC/MMP show low RGD concentration leads to faster initial proliferation.

FIG. 28. A microfluidic device (left) modified from Chung et al., Lab on a Chip 2009 (the contents of which are herein incorporated by reference in its entirety) has been established previously (Barker lab) for analysis of cell responses to gradients, engineered ECMs, etc. The device enables the analysis of complex multicellular structures such as vascular-like structures in 3D matrices (right). Preferably, in the methods disclosed herein, the cells will be cultured inside the scaffold.

FIG. 29. hES-MEC derived mesenchymal cells stabilize HUVEC networks in 3D collagen gel. A. HUVEC alone. B. HUVEC+hES-MC. [HUVEC UEA1-Red, hES-MC-GFP] 10× confocal microscopy.

FIG. 30. Porous materials were implanted subcutaneously in the dorsum of transgenic mice. The host response to the implanted material was characterized by histological analysis of collagen production and assembly (A and B) as well as visualization (C) of the implant and vessel size distribution (D).

FIG. 31. Schematic of animal studies in accordance with a preferred embodiment of the invention.

FIG. 32. Signal patterning via deprotection of the K* peptide on the 3D hydrogel. A. The caged K* peptide is inert within the hydrogel. B. a photolytic reaction via a UV lamp deprotects the K peptide in the unmasked regions of the hydrogel, exposing a free amine for functionalization of bioactive signals and RGD. C. Regions of Q-RGD functionalization to the K peptide demonstrate cell spreading, while regions containing the caged K* peptide contain cells with circular morphology. Bioactive signals, such as laminin and fibronectin, may also be patterned via the functionalization to the uncaged K peptide.

FIG. 33. K* peptide UV exposure and spacer-incorporated hydrogel chambers.

FIG. 34. Hygrogel formation through Michael addition.

### DETAILED DESCRIPTION OF THE DRAWINGS

Some embodiments of the present invention are discussed in detail below. In describing these embodiments, specific terminology is employed for the sake of clarity. The invention, however, is not intended to be limited to specific terminology so selected. One skilled in the relevant art will recognize that other equivalent components may be employed and other methods may be developed without departing from the scope of the invention. All references cited herein are incorporated by reference as if each had been individually incorporated. Headings used herein are provided for clarity and organizational purposes only, and are not intended to limit the scope of the present invention.

### DEFINITIONS

As used herein, “caged” may mean “side-chain protected” and/or “photoprotected” and/or “locked” and/or “protected” and/or “photolabile protected.”

As used herein, “photolabile protected amino acid” may mean “caged amino acid.”

As used herein, “K peptide(s)” preferably refers to a peptide sequence having K (i.e., amino acid lysine). For example, K peptide may refer to the peptide sequence FKG and/or peptide sequences having FKG, i.e., Ac-FKGGERC-NH₂. The K peptide may be identified through a rational peptide library [see, e.g., (Hu B H, Messersmith P B. Rational design of transglutaminase substrate peptides for rapid enzymatic formation of hydrogels. J Am Chem Soc 2003 Nov. 26; 125(47): 14298-14299)]. Once K peptide is “photoprotected” and/or “caged” it may be referred to herein as K* peptide and/or K Star Peptide and/or K* and/or photoprotected peptide and/or protected peptide and/or caged peptide.

As used herein, “Q peptide(s)” (i.e., amino acid glutamine) preferably refers to the peptide sequence NQEQVSPL and/or sequences containing NQEQVSPL. Preferably, the Q peptide is the sequence recognized by FXIIIa in plasminogen inhibitor α2PI. As used herein, “Q peptide-RGD” may refer to H-NQEQVSPLRGDSPG-NH₂ and/or any other sequence having Q peptide as defined herein and having the peptide sequence RGD (amino acid sequence arginine-glycine-aspartic acid). In other embodiments, Q peptide may refer to any peptide sequence having Q.

As used herein, “bioactive signal(s)” may refer to the peptide sequence RGD and/or may refer to the peptide sequence RGD linked and/or bonded with and/or attached to the Q peptide as defined therein. As such, “bioactive signal” may refer to the Q peptide-RGD sequence. “Bioactive signal” may may refer to any other bioactive signal linked and/or bonded with and/or attached to the Q peptide as defined herein. For example, it may refer to the sequence Q peptide-Fn fragment. In other embodiments, bioactive signal(s) may refer to any other bioactive signal to be immobilized/incorporated in the hydrogel using the methods of the present invention (with or without Q peptide). For example, the bioactive signal may refer to a peptide, such as, but not limited to, a peptide with a protease cleavage site. The bioactive signal may refer to fibronectin or a fragment thereof; and/or the bioactive signal may refer to a growth factor such as VEGF, or the like. Any bioactive signal capable of being immobilized in a 3D hydrogel may be used with the methods disclosed herein. It is to be understood that the bioactive signal used in a single hydrogel may be same or different.

As used herein, “substrate” or any grammatical variations thereof, preferably refers to the caged peptide and/or protein, and/or the uncaged peptide and/or protein, and/or the bioactive signal(s).

As used herein, “Fn” may refer to fibronectin and/or a fragment thereof.

As used herein, “plurality” means “one or more.”

As used herein, “placed” may refer to “immobilized.”

As used herein, “hydrogel” may be interchangeable with “hydrogel scaffold” and/or “scaffold.”

As used herein, “Factor XIII” and “FXIIIa” are interchangeable.

As used herein, “fragment” with reference to a polypeptide/peptide is used to describe a portion of a larger molecule. Thus, a polypeptide fragment may lack an N-terminal portion of the larger molecule, a C-terminal portion, or both. Fragments may include any percentage of the full-length polypeptide/peptide.

As used herein, “peptide” may refer to fragments of polypeptides and/or short polypeptides and/or polypeptides.

As used herein, “hydrogel” may refer to any optically clear polymeric network and/or any tissue engineering support system.

As used herein, the term “growth factor” includes, but is not limited to, angiogenic growth factors, such as Angiogenin, Angiopoietin-1, Del-1, Fibroblast growth factors: acidic (aFGF) and basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Proliferin, Transforming growth factor alpha (TGF-alpha), Transforming growth factor-beta (TGF-beta), Tumor necrosis factor alpha (TNF-alpha), and Vascular endothelial growth factor (VEGF)/vascular permeability factor (VPF). Other examples of angiogenic growth factors include Heparin-binding EGF-like growth factor, Interferon-gamma (IFN-gamma), Platelet factor-4 (PF-4), Macrophage inflammatory protein-1(MIP-1), Interferon-g-inducible protein-10 (IP-10), and HIV-Tat transactivating factor. The term growth factor can also include non-angiogenic growth factors such as interleukin-2 (IL-2), nerve growth factor (NGF), bone morphogenic protein (BMP), heat shock protein (HSP), and epidermal growth factor (EGF), and the like.

As used herein, the term “support” includes: natural polymeric carbohydrates and their synthetically modified, crosslinked, or substituted derivatives, such as agar, agarose, cross-linked alginic acid, chitin, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins, and keratins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass; and mixtures or copolymers of the above classes, such as graft copolymers obtained by initializing polymerization of synthetic polymers on a preexisting natural polymer. A variety of biocompatible and biodegradable polymers are available for use in therapeutic applications; examples include: polycaprolactione, polyglycolide, polylactide, poly(lactic-co-glycolic acid) (PLGA), and poly-3-hydroxybutyrate.

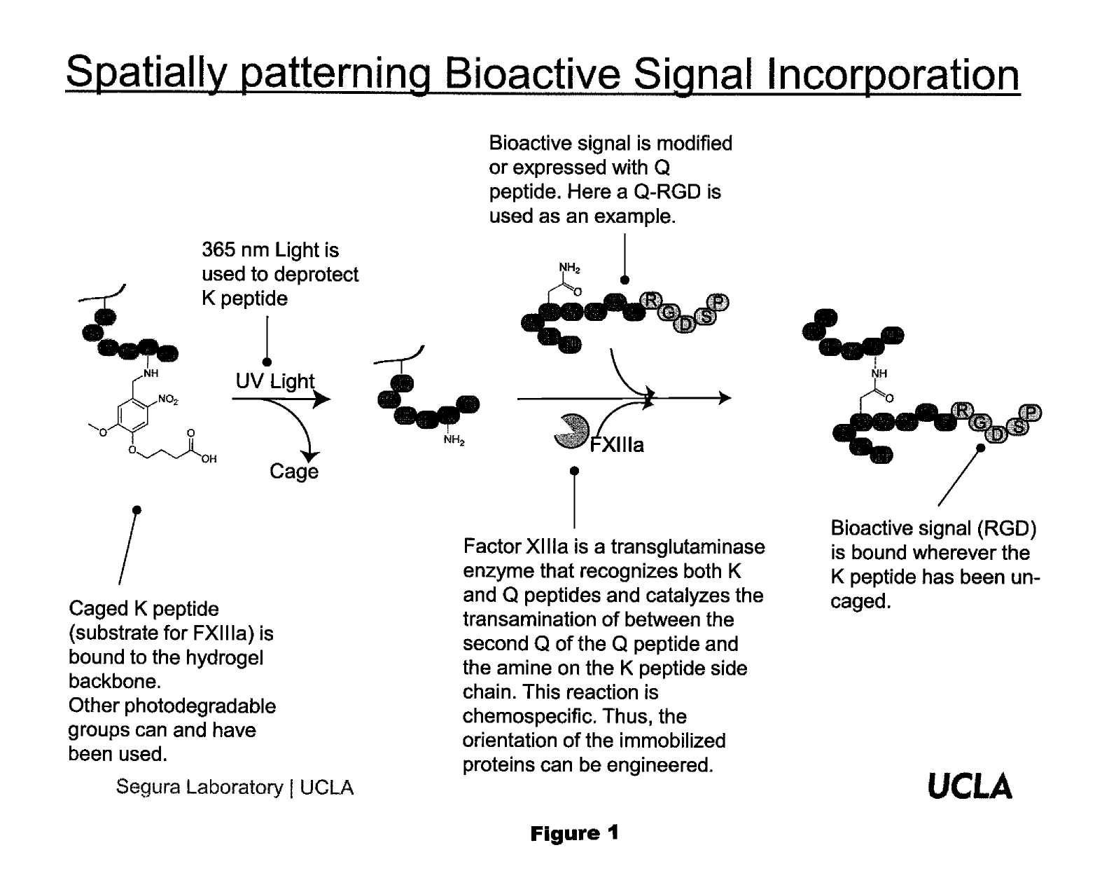

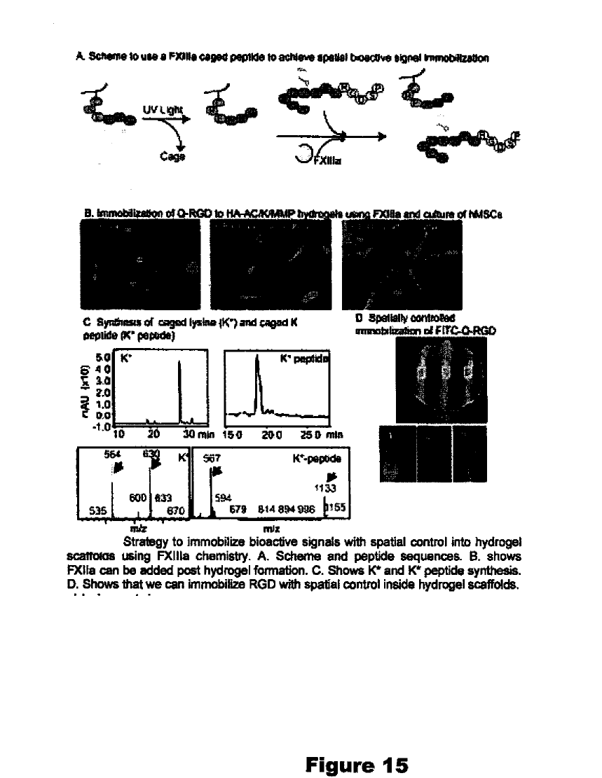

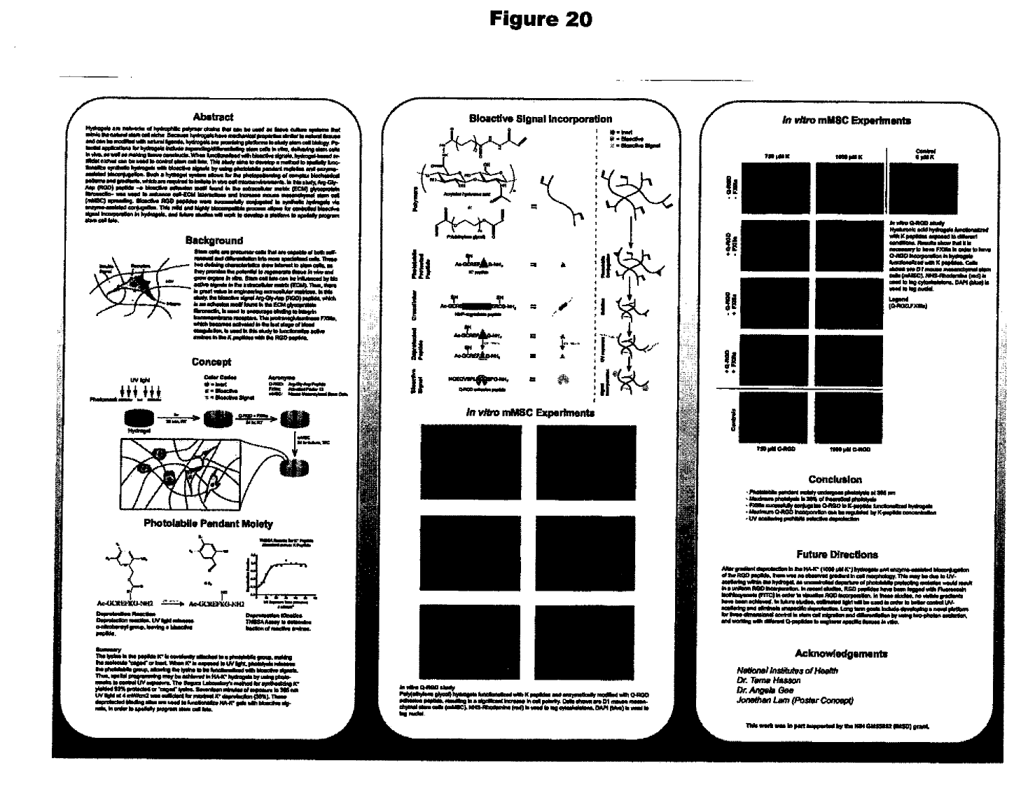

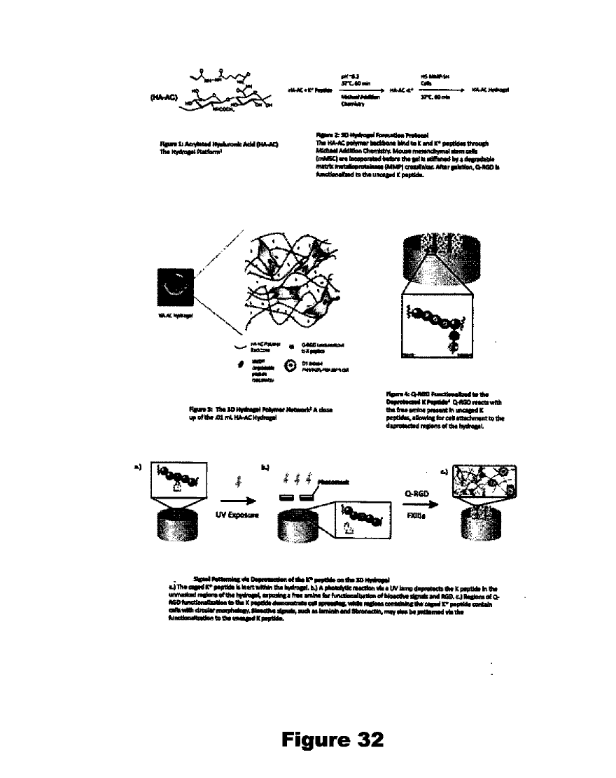

In a preferred embodiment, the present invention allows bioactive signals to be patterned in a 3D hydrogel in the following manner (as shown in FIGS. 1 and 20). A plurality of “locked” substrate(s) are added to the hydrogel before, during, and/or after hydrogel formation. Preferably, these substrates are bound to the hydrogel. The hydrogel is illuminated at certain selected locations and/or portions. Upon selective illumination, the “locked” substrate(s) in the hydrogel become “unlocked.” As such, since the hydrogel is selectively illuminated, the substrates that are illuminated become “unlocked” and the substrates that are not illuminated remain “locked.” Thereafter, the hydrogel may be incubated with an enzyme and a bioactive signal(s) (as shown in FIGS. 16 and 32). Preferably, the enzyme is able to catalyze the reaction between and/or form a bond(s) between the “unlocked” substrate and the bioactive signal(s). Preferably, the selected enzyme is unable to catalyze the reaction between and/or form a bond(s) between the “locked” substrate and the bioactive signal. In this manner, the bioactive signal is immobilized in the hydrogel only in places subjected to illumination. For example, peptides containing caged lysines (i.e., “locked lysines”) are added to the hydrogel prior and/or during and/or after hydrogel formation. Selective illumination of certain portions of the hydrogel “unlocks” these caged lysines. The system may be incubated with an enzyme such as Factor XIIIa and a peptide containing a bioactive signal such as Q-peptide-RGD. This allows Factor XIIIa to catalyze the transamination reaction between the “unlocked” lysine and the peptide containing the bioactive signal, thereby linking the “unlocked” lysine and the peptide containing the bioactive signal in a 3D hydrogel. This reaction is chemospecific. Thus, the orientation of the immobilized proteins may be engineered. The bioactive signal(s), for example, comprise certain growth factors and/or receptors and/or amino acid motifs that are known to regulate differentiation in stem cells. In this manner, the differentiation of stem cells and/or cellular migration may be controlled in processes such as tissue regeneration.

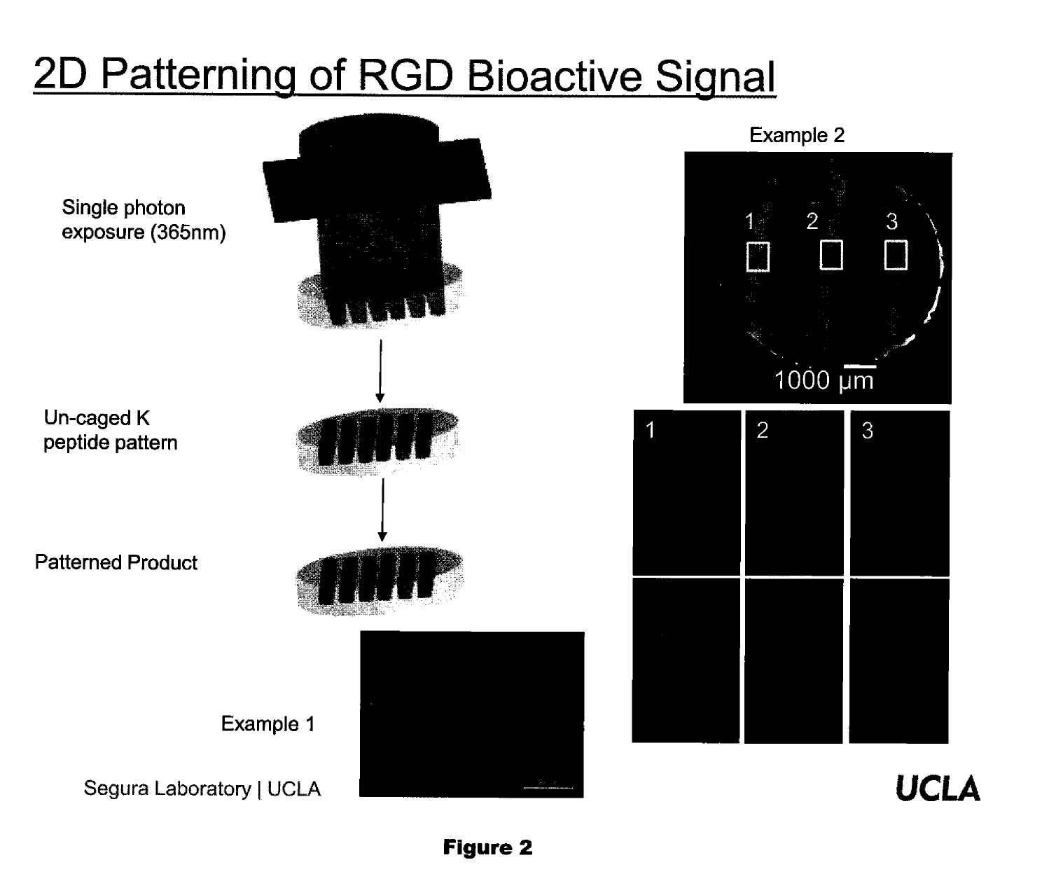

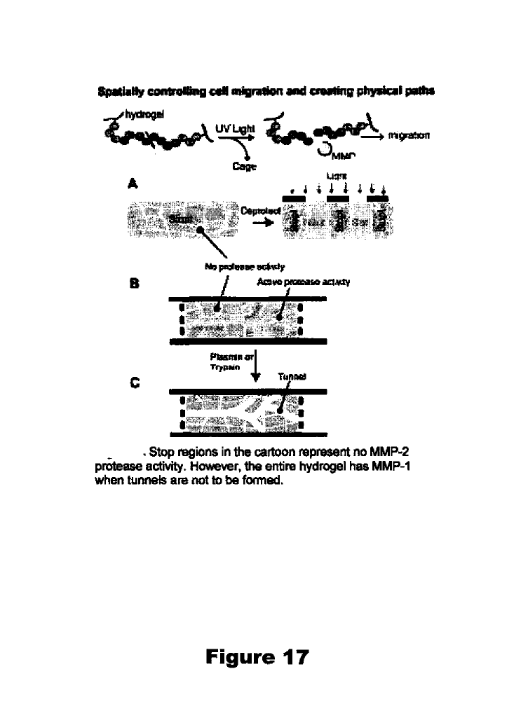

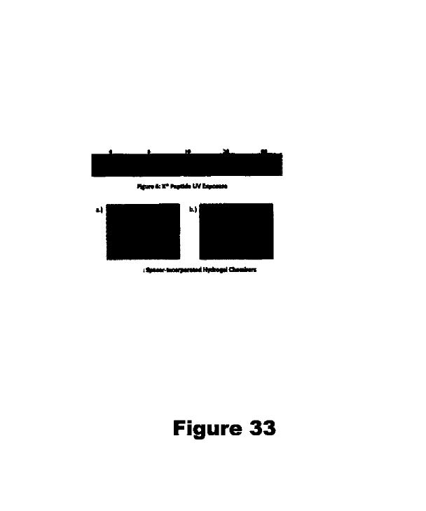

In a preferred embodiment, the bioactive signal(s) described herein, and as shown in FIGS. 1-34, are heterogeneously patterned throughout the hydrogel. It is to be understood that the bioactive signal(s) in a single hydrogel may be the same or different. For example, a first bioactive signal may be placed in a first location (i.e., at a first x, y, z coordinate) of the hydrogel, and a second bioactive signal may be placed in a second location (i.e., at a second x, y, z coordinate) of the hydrogel and/or the first bioactive signal may be placed in a first location (i.e., at a first x, y, z coordinate) of the hydrogel, and the first bioactive signal may be placed in a second location (i.e., at a second x, y, z coordinate). Preferably, the placement of these bioactive signals is heterogeneous (i.e., not evenly distributed) within the hydrogel. The spatial and temporal regulation of bioactive signals during embryonic development, adult wound healing, and stem cell niches, which occur during morphogenesis and homeostatis, demonstrate the need for the development of scaffolds with heterogeneous distribution of bioactive signals. In other embodiments, the bioactive signal(s) may be homogeneously patterned throughout the hydrogel and/or may be patterned both homogeneously and heterogeneously.

In a preferred embodiment, the system and methods described herein are used to study vascularization and/or angiogenesis. Tissue-engineered constructs are often used to replace/modify some human tissue, for example, in strokes, i.e., ischemic strokes. However, these constructs often fail, for example, as blood vessels fail to form and/or improperly form. Accordingly, there is a need to be able to control these tissue-engineered constructs so that blood vessels may properly form and/or cells may differentiate/migrate, etc. properly. As such, in the present invention, hydrogels are patterned with selected bioactive signals in selected locations and seeded with cells in order to study/control cellular differentiation and other cellular processes in tissue-engineered constructs. Furthermore, although the natural extracellular matrix (“ECM”) contains a heterogeneous composition of proteins that are presented to residing cells within discrete pockets (not a homogeneous concentration or distribution) and precise times (determined by the developmental stage of the tissue), engineered ECMs (“eECMs”) do not fully mimic this heterogeneity. Current eECMs are composed of crosslinked synthetic or natural polymers that provides the network backbone and controls the material's physical characteristics (see, e.g., Peppas, N. A.; Hilt, J. Z.; Khademhosseini, A.; Langer, R. Advanced Materials 2006, 18, 1345). The backbone polymer can be chemically modified with pendant bioactive molecules of ranging complexity, including oligopeptides (e.g. RGD adhesion peptide, see, e.g., Massia, S. P.; Hubbell, J. A. Anal Biochem 1990, 187, 292) and growth factors (e.g. vascular endothelial growth factor, see, e.g., Zisch, A. H.; Schenk, U.; Schense, J. C.; Sakiyama-Elbert, S. E.; Hubbell, J. A. J Control Release 2001, 72, 101). However, these modifications are distributed uniformly throughout the bulk of the material leading to homogeneous signal distribution that is unlike what is found in nature. Accordingly, systems/methods to provide material heterogeneity are needed. This heterogeneous patterning may allow the study of cellular developmental processes such as differentiation and/or migration.

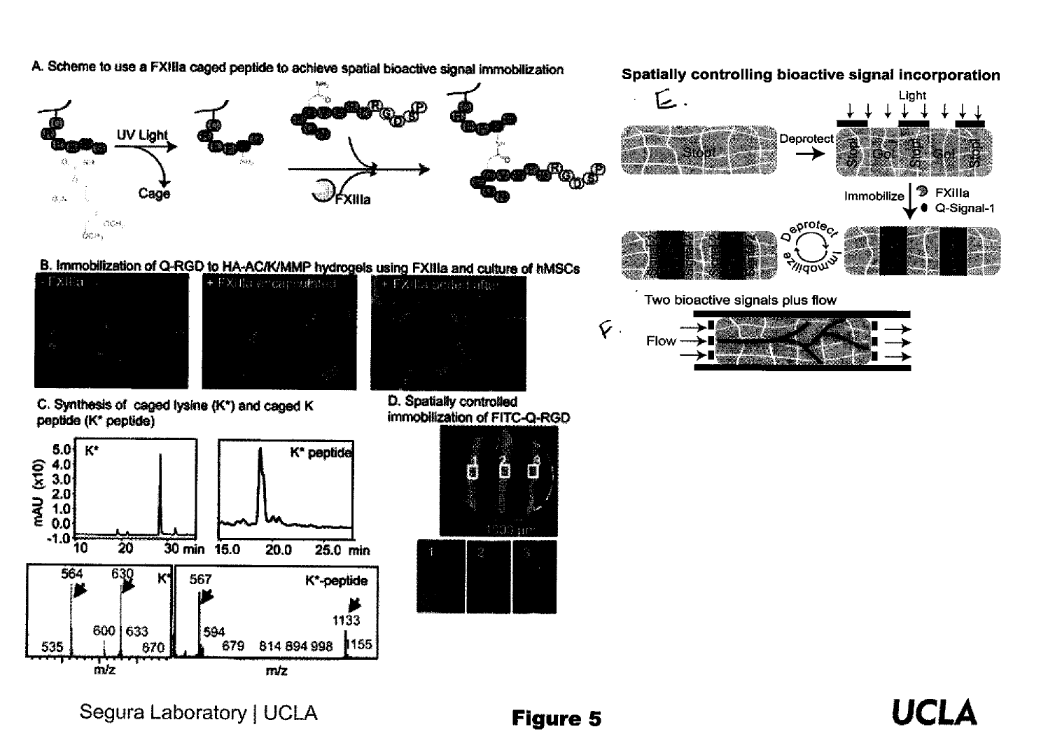

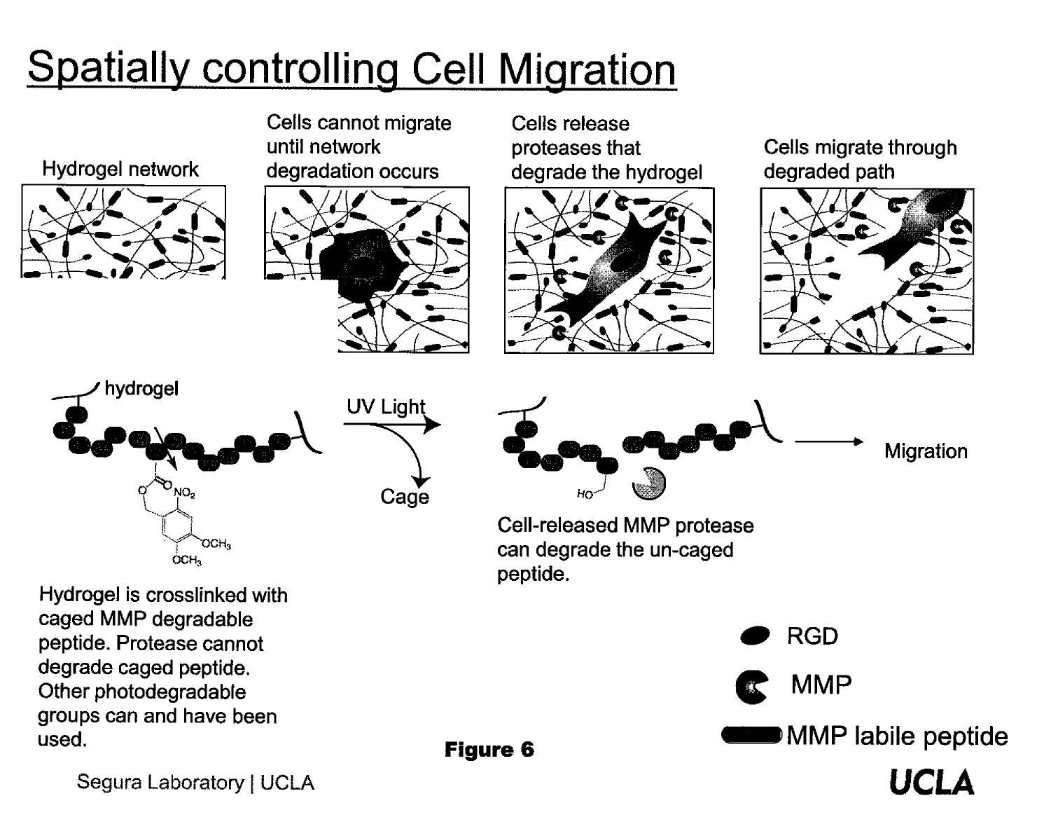

In a preferred embodiment, the patterning described above may be used with cellular migration (as shown in FIGS. 6 and 7) and/or tunneling assays described further below. For example, spacially controlled migration may affect the differentiation of cells.

### Stem Cells

In a preferred embodiment, mesenchymal stem cells (“MSCs”) are used in the invention. Preferably, these cells are used to study their differentiation into endothelial cells (“EC”) and pericyte cells (or pericyte-like cells). MSCs are a good model to study vascularization and/or angiogenesis. MSCs may be used for tissue regeneration as they are capable of being differentiated in situ into discrete epithelial and mesenchymal cellular components necessary for mature vessel formation (i.e., endothelial cells and pericytes). Preferably, differentiation into EC and pericyte cells is regulated by a process called epithelical-to-mesenchymal transition (“EMT”) [(see, e.g., Liu Z J, Zhuge Y, Velazquez O C. Trafficking and differentiation of mesenchymal stem cells. J Cell Biochem 2009 Apr. 15; 106(6):984-991)]. The EMT process is controlled by a variety of growth factors such as VEGF and PDGF, as well as integrin-specific adhesive cues from the extracellular matrix [(see, e.g., Eming S A, Brachvogel B, Odorisio T, Koch M. Regulation of angiogenesis: wound healing as a model. Prog Histochem Cytochem 2007; 42(3):115-170)]. In other embodiments, human embryonic stem cells (hESC) derived from mesodermal progenitor (hES-MEC) may be used. Preferably, mesenchymal stem cells (MSCs) are chosen for the cellular studies to demonstrate the capability of the inventive system to work in the presence of stem cells. For example, within the volumes patterned with the Q peptide, the cells responded by changing from a rounded to a more spindle-shaped morphology. In addition, the cells in the unmodified region demonstrated low cell viability. In other embodiments, any other cell line may be used, i.e., any cell line used in tissue engineering may be used.

In a preferred embodiment, the following may be used to provide spacial control in tissue differentiation and/or other cellular processes: (1) hydrogel scaffolds formed with hyaluronic acid or poly(ethylene glycol) that may be easily imaged since these materials form hydrogels that are optically clear and/or substantially clear; (2) caged amino acids that may be de-protected using long wave UV light, and (3) enzymes that recognize specific peptide sequences that can specifically cleave and/or form bonds. Each of the foregoing will be discussed in detail below.

### Optically-Clear Polymeric Networks/Hydrogels

In a preferred embodiment, an optically clear polymeric network and/or any other support network for tissue engineering applications is spatially patterned with bioactive signals using the methods disclosed herein. Suitable support materials for most tissue engineering applications are generally biocompatible and preferably biodegradable. Examples of suitable biocompatible and biodegradable supports include: natural polymeric carbohydrates and their synthetically modified, crosslinked, or substituted derivatives, such as agar, agarose, crosslinked alginic acid, chitin, substituted and cross-linked guar gums, cellulose esters, especially with nitric acid and carboxylic acids, mixed cellulose esters, and cellulose ethers; natural polymers containing nitrogen, such as proteins and derivatives, including crosslinked or modified gelatins, and keratins; vinyl polymers such as poly(ethylene glycol)acrylate/methacrylate, polyacrylamides, polymethacrylates, copolymers and terpolymers of the above polycondensates, such as polyesters, polyamides, and other polymers, such as polyurethanes; and mixtures or copolymers of the above classes, such as graft copolymers obtained by initializing polymerization of synthetic polymers on a pre-existing natural polymer. A variety of biocompatible and biodegradable polymers are available for use in therapeutic applications; examples include: polycaprolactione, polyglycolide, polylactide, poly(lactic-co-glycolic acid) (PLGA), and poly-3-hydroxybutyrate. Methods for making nanoparticles in from such materials are well-known.

In a preferred embodiment, the optically clear network and/or support network for tissue engineering applications is a hydrogel. In a preferred embodiment, a 3D hydrogel is patterned with a selected bioactive signal(s) in the present invention. Hydrogels are networks of hydrophillic polymer chains that may be used as tissue culture systems that mimic the natural stem cell niche. Because hydrogels have mechanical properties similar to natural tissues and may be modified with natural ligands, hydrogels are good platforms to study stem cell biology. Potential applications for hydrogels include differentiating stem cells in vivo, delivering stem cells in vivo, as well as making tissue constructs. Preferably, the gel is biocompatible and/or biodegradable. Biocompatible and biodegradable hydrogels, for example, find particular application in tissue engineering, where the hydrogel forms a matrix with properties sufficiently similar to extracellular matrix to permit cell and vessel migration into the matrix. Hyaluronic acid, poly(ethylene glycol), and fibrin form suitable hydrogels. Hyaluronic acid-based hydrogels can be formed from hyaluronic acid engineered, e.g., with sulfhydryl groups undergoing Michael addition with MMP-sensitive peptide diacrylates in a manner analogous to that described above. In other embodiments, the polymeric network may be substantially optically clear.

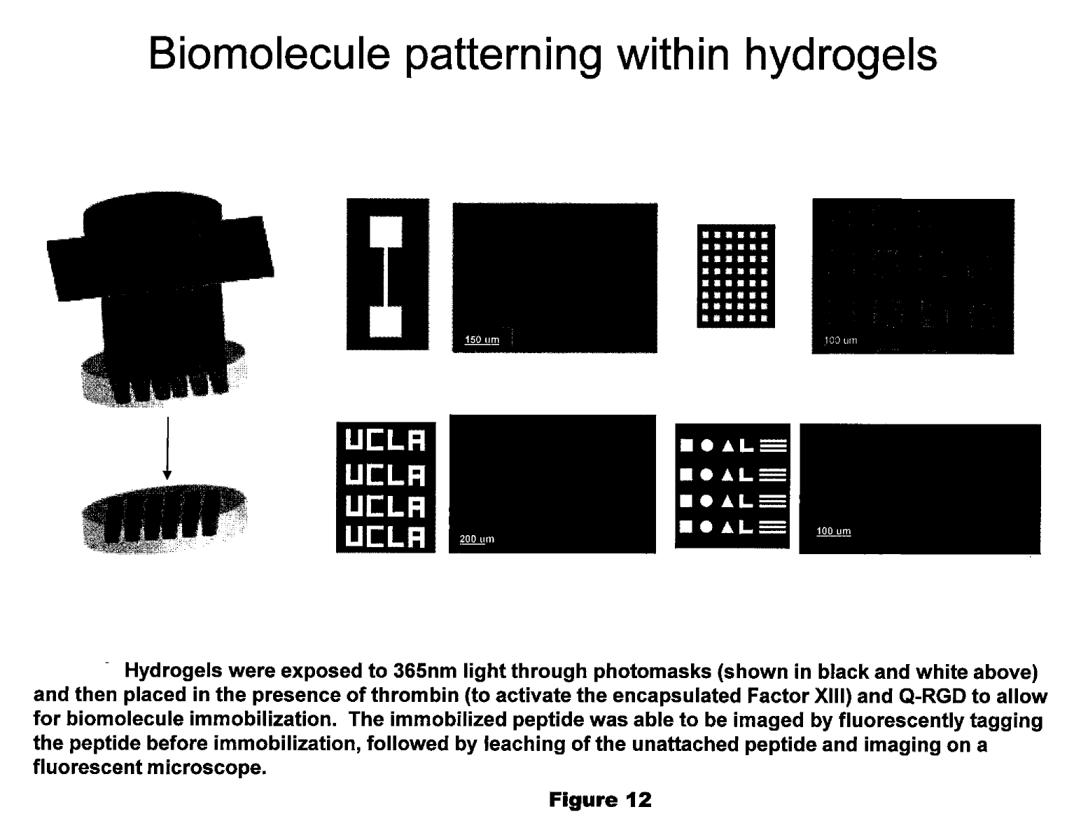

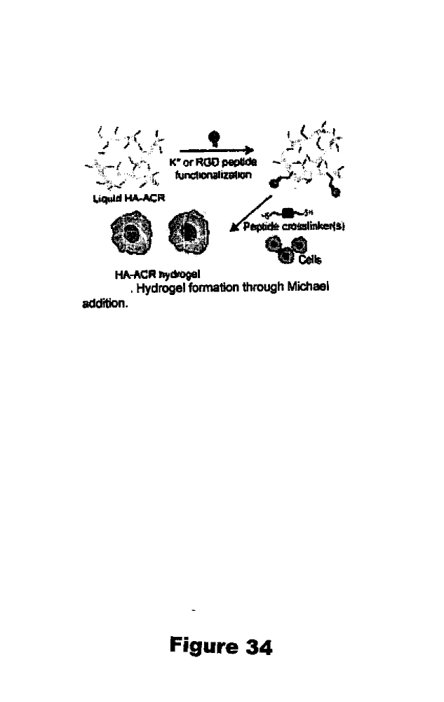

In a preferred embodiment, the hydrogel used in the present invention is a hyaluronic acid (“HA”) hydrogel. For example, hyaluronic acid-acrylate (“HA-ACR”) is used. HA is a linear disaccharide of D-glucuronate and D-N-acetylglucosamine with alternating β-1,4 and β-1,3 glycosidic bonds. HA is found in most organs and tissues, including skin, joints, and eyes [(see, e.g., Almond A. Hyaluronan. Cell Mol Life Sci 2007 May 14)]. HA and hyaluronidase (Haase) degradation fragments have also been found to be important during embryonic development, tissue organization, angiogenesis and tumorigenesis [(see, e.g., Rodgers L S, Lalani S, Hardy K M, Xiang X, Broka D, Antin P B, et al. Depolymerized hyaluronan induces vascular endothelial growth factor, a negative regulator of developmental epithelial-to-mesenchymal transformation. Circ Res 2006 Sep. 15; 99(6):583-589)]. HA is both actively synthesized and degraded into HA oligos during the initial stages of wound healing [(see, e.g., Pogrel M A, Pham H D, Guntenhoner M, Stern R. Profile of hyaluronidase activity distinguishes carbon dioxide laser from scalpel wound healing. Ann Surg 1993 February; 217(2):196-200)] and after stroke in man [(Al'Qteishat A, Gaffney J, Krupinski J, Rubio F, West D, Kumar S, et al. Changes in hyaluronan production and metabolism following ischaemic stroke in man. Brain 2006 August; 129 (Pt 8):2158-2176]). Further, HA hydrogels may promote hES stem cell renewal when unmodified with integrin binding ligands. Thus, differentiation will likely be the result of differentiation signals introduced into the scaffold. Accordingly, the HA hydrogel is an ideal scaffold to transplant cells into the brain after stroke and to aid in wound healing. HA hydrogels may promote hES stem cell renewal when unmodified with integrin binding ligands [(see, e.g., Gerecht, S. et al. Hyaluronic acid hydrogel for controlled self-renewal and differentiation of human embryonic stem cells. Proc Natl Acad Sci USA 104, 11298-11303 (2007)]. As such, preferably, differentiation will be the result of signals introduced into the scaffold. Unlike poly(ethylene glycol) (“PEG”), the HA hydrogel may be customized to contain more sites of modification and/or cross-linking, and may be completely biodegradable. Additionally, the hydrogels used in the present invention may be prepared through initial incubation with the photoprotected peptide with the backbone polymer, followed by addition of an MMP-degradable peptide with cysteines. In other embodiments, a PEG hydrogel may be used. For example, PEG hydrogel may be available as 2, 4, or 8 arm molecules and, thus, may provide a maximum of 8 sites for modification and/or crosslinking per molecule. [(The HA hydrogel, however, may be modified to contain about 77 acrylate groups per molecule. Accordingly, it may provide at least 9 times more sites for modification and/or crosslinking. Preferably, having more sites available for modification/crosslinking results in a wider range of bioactive signal incorporation without compromising crosslinking density (i.e., mechanical properties). For example, with even 77 acrylates per chain (48% modification of the COOH groups in HA), HA hydrogels may be completely degradable by hyaluronidase)]. For example, PEG-vinyl sulfone (“PEG-VS”) may be used. PEG is a synthetic polymer that is widely used in biomedical applications ranging from implant coatings to drug delivery and tissue engineering. Because PEG is biologically inert, it can serve as a blank slate to display bioactive signals and study their role in stem cell differentiation or renewal. Further, both HA and PEG polymers are highly hydrated in water and have low protein absorption to their backbone, which is ideal for the synthesis of biomaterials with a very defined composition. The synthetic approach used to crosslink HA and PEG polymers into hydrogels must allow for the encapsulation of stem cells. Thus, it may be done under close to pH=7.4, 4° C. to 37° C. temperature, and in aqueous buffers with 150 mM salt. For example, Michael addition of dithiol containing crosslinkers to vinyl groups present on HA or PEG may be used to crosslink the networks (as shown in FIGS. 13 and 34) [(see, e.g., Adelow C, Segura T, Hubbell J A, Frey P. The effect of enzymatically degradable poly(ethylene glycol) hydrogels on smooth muscle cell phenotype. Biomaterials 2008 January; 29(3):314-326)]. Prior to crosslinking, the HA-ACR or PEG-VS may be modified with integrin binding peptides such as RGD if no ECM proteins are to be incorporated and the K* peptide via Michael addition. In yet other embodiments, any hydrogel and/or hydrogel-based system may be used in the invention.

### Substrates

In a preferred embodiment, one or more substrates are used in the present invention. These substrates may be added to the hydrogel before and/or during and/or after its formation. Preferably, these substrates are “locked” and “unlocked” via illumination. Preferably, the substrate is a caged amino acid and/or a peptide containing a caged amino acid and/or a caged peptide (which becomes uncaged upon illumination). For example, the substrate may be a bioactive signal(s). Caged amino acids have been used to block enzymatic activity, peptide-receptor interactions, and growth factor activity [(see, e.g., (Lawrence, D S. The Preparation and in vivo applications of caged peptides and proteins. Curr Opin Chem Biol 2005 Dec. 9(6):570-575; and Shigeri, Y.; Tatsu, Y.; Yumoto, N. Pharmacol Ther 2001, 91, 85)]. Preferably, caged amino acids that are de-protected using long wave UV light (preferably 365 nm) are used. Preferably, one or more of the following may be used as a substrate and/or caged amino acid in the present invention: lysine (K), aspartic acid (D), glutamic acid (E), arginine (R), serine (S), tyrosine (Y), and/or cysteine (C). Most preferably, the caged amino acid used in the present invention is lysine (k). In other embodiments, any other amino acid may be used as a caged amino acid. In other embodiments, the substrate may be one or more peptide sequences and/or carbohydrates, and/or small molecules and/or combinations and/or mixtures thereof. For example, the substrate may be a peptide, i.e., a peptide having a protease cleavage site and/or a peptide degradable by a protease such as MMP-2.

In a preferred embodiment, the caged amino acids may be provided in the form of a peptide wherein one or more of the amino acids are caged. In other embodiments, caged peptides may be provided. For example, peptides having a protease cleavage site and/or peptide degradable by a protease may caged and bonded/linked to the hydrogel, i.e., a peptide degradable by plasmin and/or trypsin and/or a peptide degradable by, for example, MMP-2 may be caged. It is to be understood that any other peptide may be caged, as long as it is uncaged upon illumination.

In a preferred embodiment, the caged amino acid is lysine (“K”). Preferably, the peptide having K (“K peptide”) is Ac-FKGGERC-NH₂. In other embodiments, any other peptide sequences containing K may be used as substrates in the present invention. In yet other embodiments, any other peptide sequences containing aspartic acid (D), glutamic acid (E), arginine (R), serine (S), tyrosine (Y), and/or cysteine (C) may be used as substrates in the present invention.

In a preferred embodiment, the K peptide may be synthesized to contain an ortho-nitrobenzyl (o-NB) photocaged lysine in order to achieve spatial control over bioactive signal incorporation [(see, e.g, Griffin D R, Kasko A M J. Am. Chem. Soc., 2012, 134 (42), pp. 17833-17833)]. This moiety has been used to cage free thiol groups in a hydrogel [(see, e.g., Wylie, R. G.; Ahsan, S.; Aizawa, Y.; Maxwell, K. L.; Morshead, C. M.; Shoichet, M. S. Nat Mater 2011, 10, 799)]. The o-NB protecting group intermediate [i.e., 4-(4-formyl-2-methoxy-5-nitrophenoxyl) butanoic acid] may be prepared by following a previously established protocol [(see, e.g., Griifin D R, Kasko A M J. Am. Chem. Soc., 2012, 134 (42), pp. 17833-17833)]. This caged peptide may be immobilized into the bulk of the hydrogel. Preferably, the o-NB is used to “lock” the peptide to enzymatic activity and UV light is used to “unlock” the peptide. Enzymatic action may only occur in regions that have been exposed to UV light. Since the o-NB photoprotecting group degrades under the same cell compatible UV light conditions used for photoencapsulation of cells (λₑₓ>350 nm) [(see, e.g., Griffin, D. R.; Kasko, A. M. J Am Chem Soc 2012; Bryant, S. J.; Nuttelman, C. R.; Anseth, K. S. J. Biomater. Sci.-Polym. Ed. 2000, 11, 439)], the caged K-peptide can be deprotected under cell compatible conditions. In yet other embodiments, amino acids besides lysine may be synthesized to contain an o-NB photocaged amino acid in order to achieve spatial control over bioactive signal incorporation. In yet other embodiments, the K peptide may not be synthesized to contain an o-NB photocaged lysine.

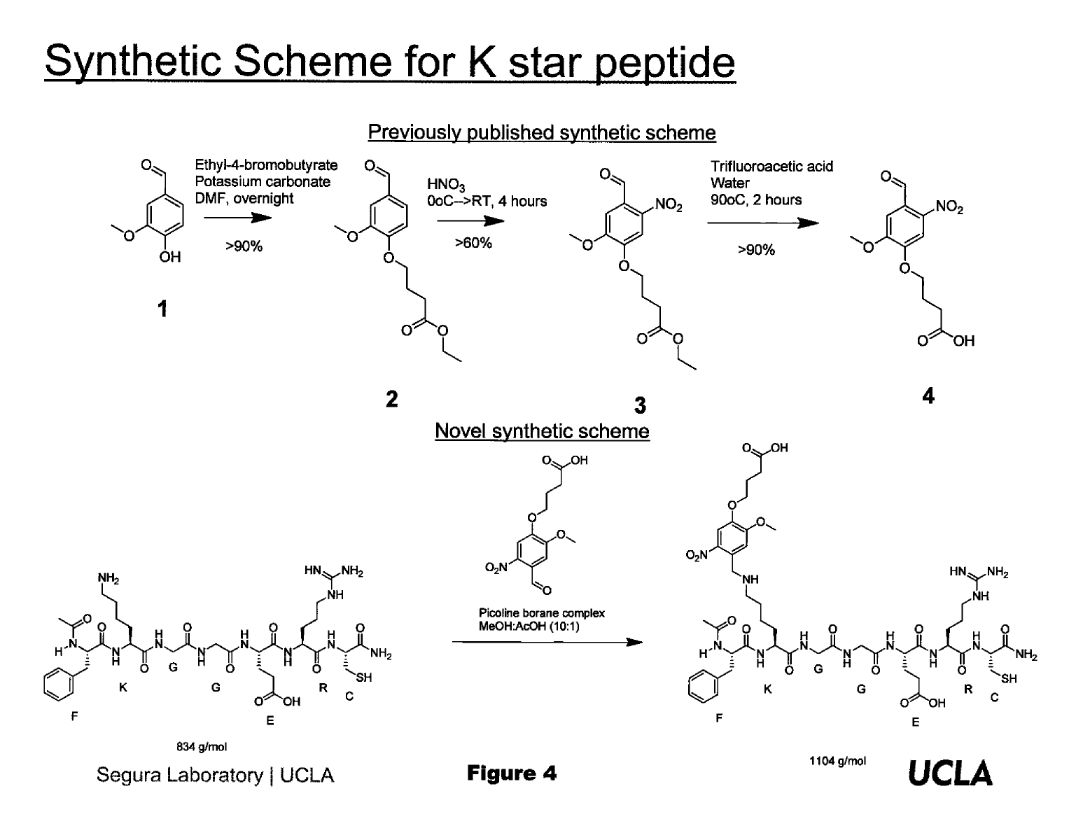

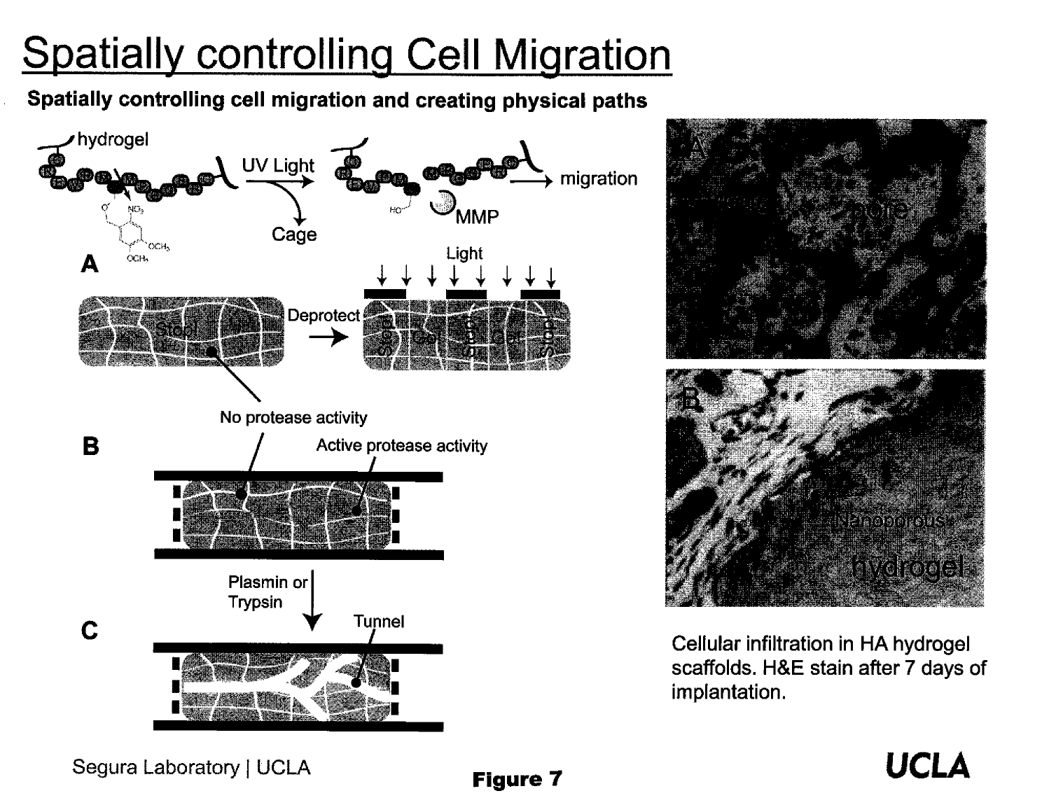

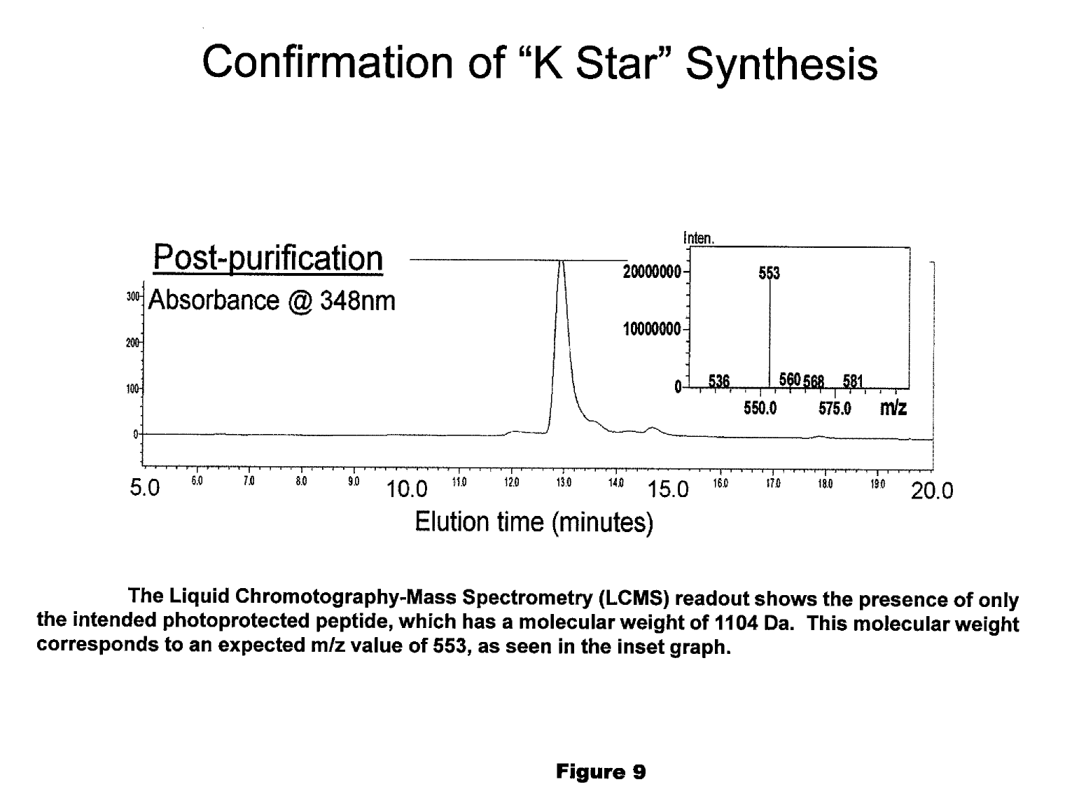

In a preferred embodiment, the photoprotected peptide with o-NB is prepared as follows (as shown in FIGS. 4 and 8). The K peptide may be reacted with the o-NB protecting group intermediate via a reductive amination that results in the formation of a highly stable secondary amine linkage. The disappearance of the starting peptide and emergence of the photoprotected peptide may be observed by liquid chromatography-mass spectroscopy or other known methods. Preferably, the photoprotected peptide is purified by preparatory high-performance liquid chromatography (HPLC), or other known methods, to yield a pure sample. Following purification, K Star may be stored in an aqueous environment at 4° C. for about six weeks without any noticeable loss of purity. Preferably, the photodegradation kinetics of K Star may be tested in the following manner. A solution of the K Star peptide may be exposed to UV light (λₑₓ=365 nm, I₀=20 mW/cm². For example, upon exposure to these conditions the immobilized peptide may be 50% deprotected after ˜3 minutes (as shown in FIG. 10). Due to the predictable nature of photodegradation, degradation data may be used to produce a predictive equation (as shown in FIG. 10) that uses 365 nm light intensity and exposure duration to predict expected deprotection.

In a preferred embodiment, the photoprotected peptide may be synthesized by preparing a caged lysine K amino acid and then using solid phase peptide synthesis to make the protected peptide (as shown in FIG. 13).

In a preferred embodiment, any photoactive caging group that may be removed and/or deprotected and/or unlocked via illumination to reveal an active substrate for enzyme interaction may be used without departing from the scope of the present invention. For example, other 2-nitrobenzyl derivatives; 7-nitroindoline derivatives; coumarin-4-methyl derivatives; para-hydroxyphenacyl derivates [(see, e.g., Conic, J. E. T.; Furuta, T.; Givens, R.; Yousef, A. L.; Goeldner, M. In Dynamic Studies in Biology; Wiley-VCH Verlag GmbH & Co. KGaA: 2005, p 1)].

In a preferred embodiment, any photoactive caging group described herein may be attached via any chemistry that will not permanently alter the enzyme substrate in such a way that makes the substrate inactive once the photocage is removed via illumination. For example one or more of the following chemistries may be used: lysines—reductive amination; carbamate formation; glutamic acid and aspartic acid—esterification by carbodiimide or N-hydroxysuccinimide coupling [(see, e.g., Basle, E.; Joubert, N.; Pucheault, M. Chemistry & amp; Biology 2010, 17, 213)].

In a preferred embodiment, the photocaging group may be added before and/or after and/or during synthesis of the substate, i.e., the photocaging group may be added pre-synthesis (i.e. photocage attached before the complete formation of the enzyme substrate) and/or post-synthesis (i.e., photocaging group added after complete formation of the substrate).

### Bioactive Signal(s)

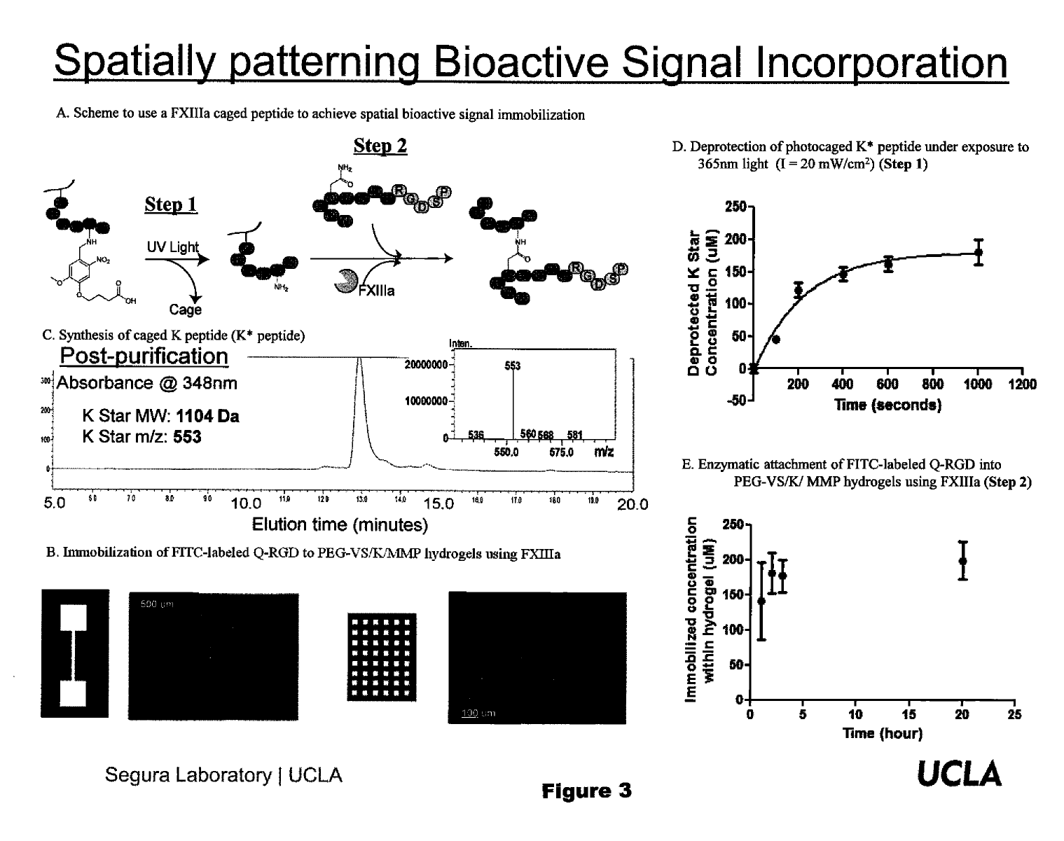

In a preferred embodiment, the hydrogels disclosed herein are immobilized with one or more bioactive signals. It is to be understood that one or more different bioactive signals may be immobilized within a single hydrogel. The bioactive signal may be linked with Q-peptide; however, it does not need to be. The bioactive signal(s) as disclosed herein may be one or more growth factors, extracellular matrix proteins, peptides, carbohydrates and/or a fragment(s) thereof. Preferably, the bioactive signal(s) in the present invention is RGD (or the Arg-Gly-Asp peptide) (as shown in FIGS. 1 and 3). RGD is a bioactive adhesion motif found in the extracellular matrix (ECM) glycoprotein fibronectin. Preferably, RGD is an integrin-binding site and/or intergrin-specific Fn fragment that is capable of directing spatially-controlled differentiation of certain stem cells into endothelial and pericyte-like cells. Stem cell fate may be influenced by bioactive signals in the extracellular matrix.

In a preferred embodiment, the bioactive signal(s) in the present invention is one or more integrin-specific Fn fragments. For example, intergrin-specific Fn fragments play a role in directing vasculogenesis and angiogenesis during embryonic development and adult wound healing via integrin-specific signal transduction and/or temporal developmental integrin switches, particularly with α-5 and β1 integrins. Additionally, these Fn fragments may be engineered to bind αvβ3 and/or α5β1 with great specificity leading to intergrin-specific differentiation of stem cells and that these fragments are capable of directing EMT in epithelial precursor cells [(see, e.g., Martino, M. M. et al. Controlling intergrin specificity and stem cell differentiation in 2D and 3D environments through regulation of fibronectin domain stability. Biomaterials 30, 1089-1097 (2009)]. More than about 20 different intergrin heterodimers exist. Their engagement within the ECM determines cell behaviors ranging from proliferation and apoptosis to migration and differentiation. For this reason, the specificity of intergrin binding during the life cycle of the cell is regulated. Fibronectin's role in directing cell and tissue homeostasis and repair is through the binding and activation of integrins and predominantly occurs through the RGD (Arg-Gly-Asp) recognition sequence located on the 10^{th} type III repeat. The recognition of this tripeptide sequence may depend on flanking residues, its three dimensional presentation, and/or individual features of the integrin-binding pockets.

In a preferred embodiment, the bioactive signal(s) may be RGD in concert and/or linked with second recognition sequence (PHSRN), the so-called “synergy” site, in the adjacent 9^{th} type repeat that is known to promote the specific interaction of α5β1 intergrin binding to Fn through interactions with the α5 subunit [(see, e.g., Mardon, H. J. & Grant, K. E. The role of the ninth and tenth type IIII domains of human fibronectin in cell adhesion. FEBS letters 340, 197-201 (1994); and Mould, A. P. et al. Defining the topology of integrin alpha5beta1-fibronectin interactions using inhibitory anti-α5 and anti-beta 1 monoclonal antibodies. Evidence that the synergy sequence of fibronectin is recognized by the amino-terminal repeats of the alpha-5 subunit. The Journal of Biological Chemistry. 272, 17283-17292 (1997)]. Additionally, α3β1 may be promoted by the 9^{th} type repeat. The spatial orientation, and thus the synergistic activity, of the RGD and synergy cell adhesion peptides may be highly sensitive to mechanical forces generated by resident cells. As such, it is to be understood that the bioactive signal may be RGD in concert with synergy. In this manner, Fn fragments may be able to leverage synergy and RGD spacial orientation to modulate αv, α5, and/or α3 intergrin binding. It is to be understood, however, that the bioactive signal RGD does not have to be provided in concert with synergy. It may act on its own as a bioactive signal and/or be attached to other peptides and/or proteins of interest without departing from the scope of the present invention.

In a preferred embodiment, the bioactive signal is engineered depending on the type of integrin binding desired. For example, Fn conformation is sensitive to mechanical forces, a fact that is exemplified into its own polymerization into fibrillar form. These conformation changes may be naturally driven by the input of cellular energy in the form of cell contractule forces. The type-III repeats that comprise the cell-binding domain (7^{th}-10^{th} type III repeats) within Fn are particularly sensitive to mechanical forces since these individual domains are stabilized only by van der Waals forces and hydrogen bonding between amino acid side chains of opposing beta sheets. Due to their elasticity, the 9^{th} and 10^{th} type III repeats together present multiple conformations that direct integrin specificity to this region. Preferably, in their native conformation, the synergy site is located at 32 Å from RGD and the two motifs act synergistically to bind α5β1 intergrin. Simulations and modeling indicate that in response to small forces (less than 100 N) the Synergy-RGD distance increases to about 55 Å, a distance too large for both sites to co-bind α5β1. For example, there may be an inverse relationship between the length of the linker chain between the two type-III repeats and α5β1 binding. As such, the α5β1 binding attributed to the Synergy site may be turned off mechanically by stretching these domains into the intermediate state or beyond. Furthermore, a stabilization of the 9^{th} type-III repeat via a Leu to Pro point mutation at amino acid 1408 or stabilization of the hydrogen bonding within the 10^{th} type-III repeat increases affinity for α5β1 over αvβ3.

In a preferred embodiment, the bioactive signal is not RGD. For example, any bioactive signal that contains a Q containing peptide and/or linked to a Q containing peptide sequence recognized by FXIIIA may be immobilized with spacial control. Laminin and fibronectin both have FXIIIa recognition sequences and can be immobilized to the hydrogel surface without further modification. In yet other embodiments, the bioactive signal is RGD in connection with any other bioactive signals.

In a preferred embodiment, the bioactive signal may be one or more growth factors. For example, the bioactive signal may be Q-VEGF and/or Q-PDGF (Q peptide with the respective growth factors). In yet other embodiments, the bioactive signal(s) does not contain the Q-peptide as disclosed herein. For example, the bioactive signal(s) may be immobilized without the use of the Q-peptide as disclosed herein, without departing from the scope of the present invention. In yet other embodiments, the bioactive signal may be protein fragments such as carbohydrates (i.e., heparin), small molecule drugs, and/or synthetic polymers and/or any extracellular matrix protein (i.e., collagen, fibronectin, laminin, vitronectin, and fibrin), peptide (i.e., adhesion moieties (RGD, IKVAV), antimicrobial peptides), carbohydrate (hyaluronic acid) or/or any fragment of thereof and/or any combination thereof.

In a preferred embodiment, the biosignal(s) described herein may be attached to the substrate via solid phase synthesis (i.e., for peptide biosignals); via DNA cloning (i.e., for protein biosignals); via NHS-ester conjugation chemistry; thiol-ene conjugation chemistry and/or disulfide attachment.

Q peptide

In a preferred embodiment, “Q peptide(s)” preferably refers to the peptide sequence NQEQVSPL and/or sequences containing NQEQVSPL (as shown in FIG. 1). The Q peptide is the sequence recognized by FXIIIa in plasminogen inhibitor α2P1 (see, e.g., Sakata Y, Aoki N. Cross-linking of alpha 2-plasmin inhibitor to fibrin by fibrin-stabilizing factor. J Clin Invest 1980 February; 65(2):290-297)]. As used herein, “Q peptide-RGD” may refer to H-NQEQVSPLRGDSPG-NH₂ and/or any other sequence having Q peptide as defined herein and having the sequence RGD. In other embodiments, Q peptide may refer to any peptide sequence having Q. Preferably, the Q peptide is linked to the bioactive signal(s). In other embodiments, the bioactive signal may be immobilized without the use of the Q-peptide.

### Enzymes

In a preferred embodiment, an enzyme that may cleave the selected substrate(s) to produce a site to which a bioactive signal is subsequently attached and/or to form a bond(s) between the selected substrate and the selected bioactive signal is used in the present invention. Preferably, the selected enzyme forms a bond between the selected substrate(s) and the selected bioactive signal(s). Preferably, the bond is a covalent bond. In other embodiments, the bond is not a covalent bond. It is to be understood that any enzyme that is capable of covalent bond formation and/or any other bond may be used. Preferably, the invention disclosed herein includes the use of any enzyme capable of covalent bond formation and the photoactive caging of one of two enzyme-recognized substrates that participate with said enzyme.

In a preferred embodiment, the enzyme Factor XIIIa (or “FXIIIa”) is used in the present invention. FXIIIa is a naturally-occurring transglutaminase enzyme that catalyzes the formation of a covalent bond between K and Q amino acids in proteins or peptides. Specifically, it catalyzes a transamination reaction between the second Q of the Q peptide described herein and the amine on the K peptide side chain to generate a non-canonical covalent bond between the Q and the K amino acid side chains. Preferably, this reaction is chemospecific. FXIIIa and the peptide NQEQVSPL (derived from 2-plasmin inhibitor (₂-PI₁₋₈) [(Schense, J. C.; Hubbell, J. A. Bioconjug Chem 1999, 10, 75)] have been previously used to immobilize growth factors [(see, e.g., Zisch A H, Schenk U, Schense J C, Sakiyama-Elbert S E, Hubbell J A. Covalently conjugated VEGF—fibrin matrices for endothelialization. J Control Release 2001 May 14; 72(1-3):101-113)], protein fragments [(see, e.g., Martino M M, Mochizuki M, Rothenfluh D A, Rempel S A, Hubbell J A, Barker T H. Controlling integrin specificity and stem cell differentiation in 2D and 3D environments through regulation of fibronectin domain stability. Biomaterials 2009 February; 30(6):1089-1097)] and peptides [(see, e.g., Schense J C, Hubbell J A. Cross-linking exogenous bifunctional peptides into fibrin gels with factor XIIIa. Bioconjug Chem 1999 January-February; 10(1):75-81)] to fibrin hydrogels through bulk modification. Further, in combination with the peptide GCE-FKG, FXIIIa has been used to catalyze the gelation of PEG to form a hydrogel [(see, e.g., Ehrbar M, Rizzi S C, Schoenmakers R G, Miguel B S, Hubbell J A, Weber F E, et al. Biomolecular hydrogels formed and degraded via site-specific enzymatic reactions. Biomacromolecules 2007 October; 8(10):3000-3007; Hu, B. H.; Messersmith, P. B. J Am Chem Soc 2003, 125, 14298)]. In nature, this enzyme is active during the wound-healing cascade, where it stabilizes fibrin clots and introduces bioactive signals to the clot such as fibronectin, collagen, and laminin. In other embodiments, other enzymes may be used to catalyze a reaction and/or form a bond between the Q-peptide-RGD and the K peptide. In other embodiments, any other transglutamase enzyme may be used. For example, one or more of the following may be used in lieu of, or in connection with, FXIIIa: transglutamases 1-7 and/or any other enzyme capable of forming an amide bond between a lysine and a glutamine may be used. In yet other embodiments, any enzyme capable of catalyzing/forming covalent bonds may be used in lieu of, or in connection with, FXIIIa. For example, the bioactive signals disclosed herein may be covalently attached to an enzyme-recognized substrate via any method, including synthetic chemistry methods (i.e., peptide synthesis methods), DNA cloning, and conjugation chemistry.

Preparation of Hydrogels with Substrate(s)

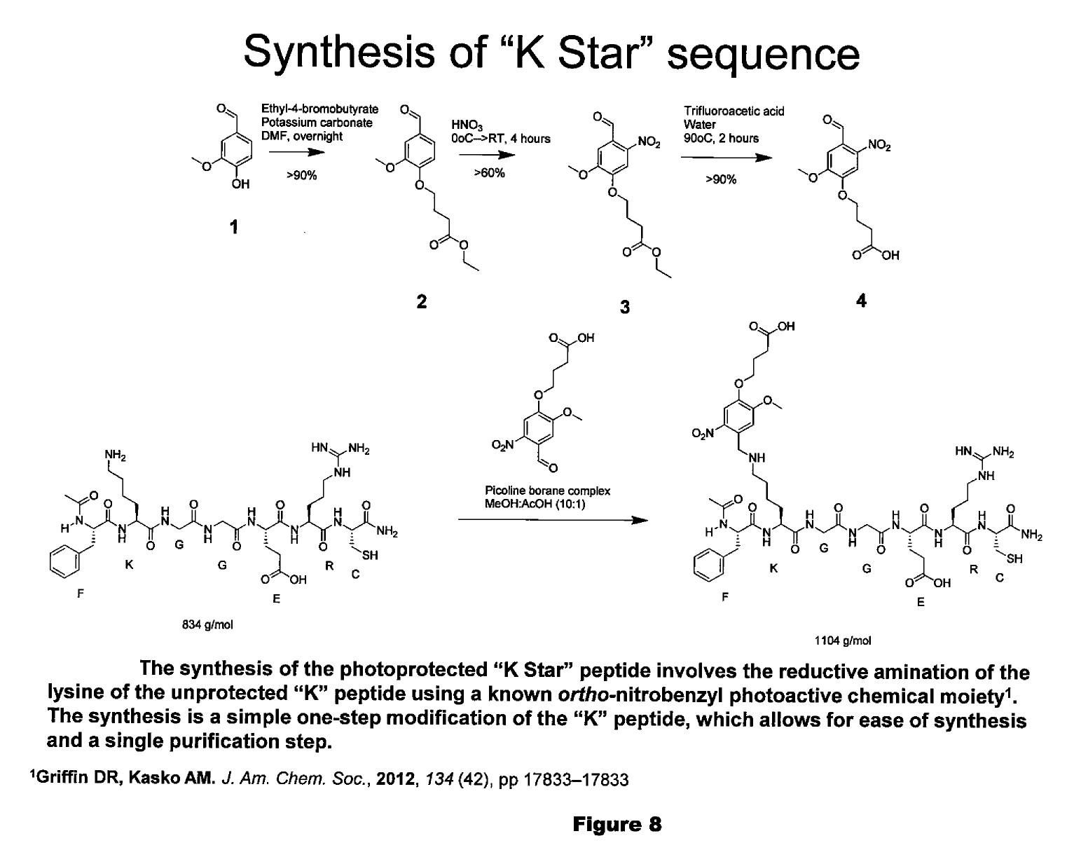

In a preferred embodiment, the hydrogel may be modified with the K* peptide prior to hydrogel formation. Preferably, K* synthesis may be confirmed using liquid chromatography mass spectrometry (as shown in FIG. 9). Preferably, the optimal concentration of K* peptide is determined using radiolabeled K* peptide and radiolabeled proteins. Preferably, the hydrogel is synthesized inside a microfluidic device mouted on a glass coverslip with or without cells embedded in the hydrogel. In other embodiments, the hydrogel may be modified with the K* peptide and/or any other caged amino acid/peptide prior to and/or during and/or after hydrogel formation.

Preparation of Hydrogels with Inactive Enzyme(s)

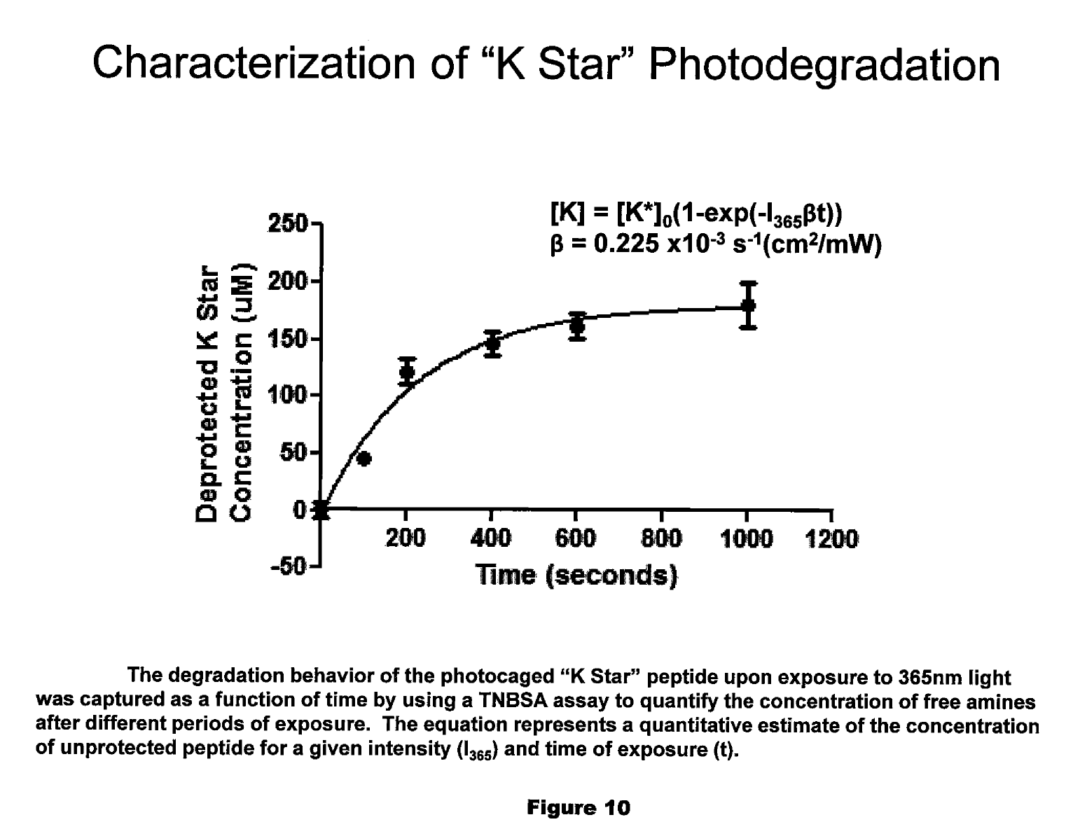

In a preferred embodiment, the selected enzyme may be added and/or included to the hydrogel before, during, and/or after hydrogel formation. Preferably, the selected enzyme (i.e., Factor XIII) is included within the hydrogel during gelation in its inactive form. The inactive enzyme may be included within the hydrogel to eliminate and/or reduce diffusion time of the larger, active Factor XIII (about 140 kDa) through the hydrogel network, which allows for more uniform patterning. Additionally, adding the inactive enzyme allows for delayed patterning as the inactive enzyme is highly stable and may be activated by subsequent addition of thrombin (a much smaller protein, about 35 kDa). This method shows high efficacy for attachment of biomolecules within hydrogels containing either the unmodified K peptide or the K star peptide (as shown in FIG. 11). In addition, the predicted immobilization matches very well with the actual immobilization for the exposed K star hydrogels. In other embodiments, the inactive enzyme and/or the inactive Factor XIII is not added to the hydrogel before, during, and/or after hydrogel formation.

### Illumination

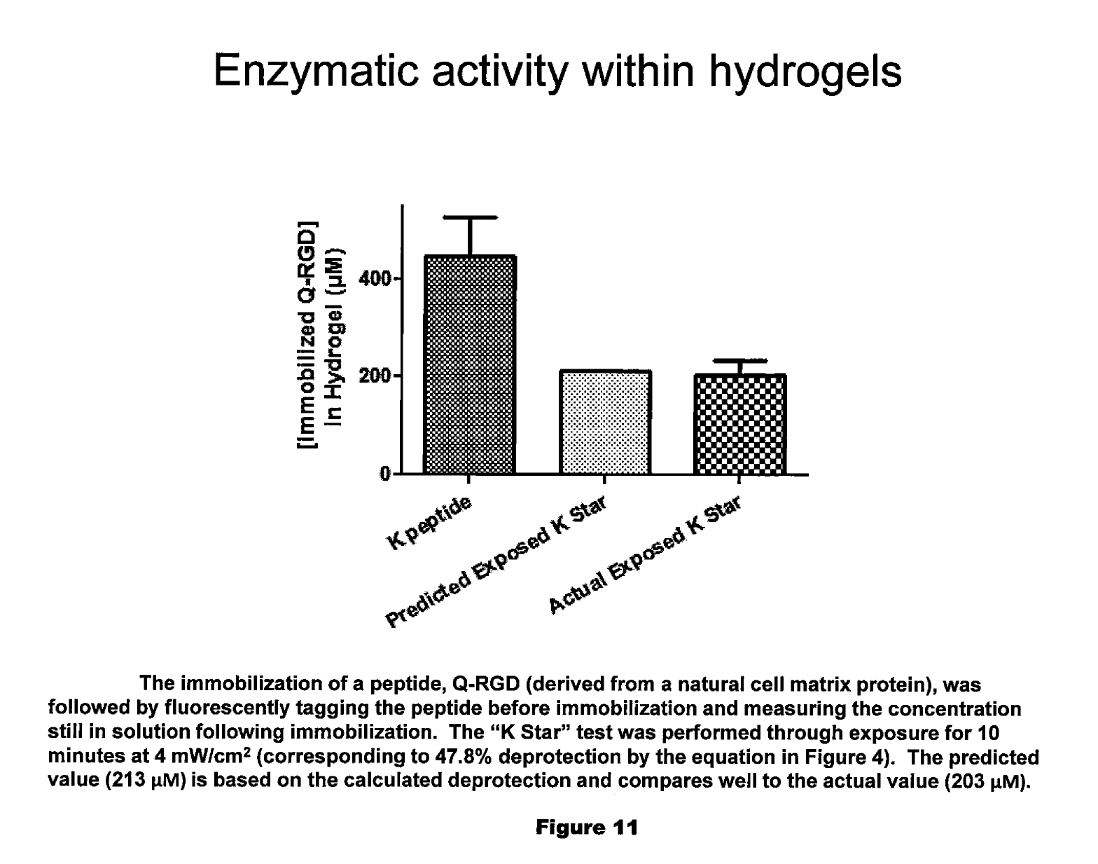

In a preferred embodiment, the hydrogel may be illuminated after being modified with the substrate(s). Preferably, a two-photon confocal microscope [(see, e.g., Lee S H, Moon J J, West J L. Three-dimensional micropatterning of bioactive hydrogels via two-photon laser scanning photolithography for guided 3D cell migration, Biomaterials 2008 July: 29(20):2962-2968)] or a photomask and a UV light source is used to “deprotect” the substrate(s) (e.g., the K* peptide). Preferably, the UV light source is 365 nm (4 mW/cm²). For example, hydrogels may be exposed to 365 nm light through photomasks, and then placed in the presence of thrombin (to activate the encapsulated Factor XIII) and Q-RGD to allow for biomolecule immobilization (as shown in FIG. 12). Thereafter, the immobilized peptide may be imaged by fluorescently tagging the peptide before immobilization, followed by leaching of the unattached peptide and imaging on a fluorescent microscope. In other embodiments, the UV light source is less than about 365 nm or more than about 365 nm.

In a preferred embodiment, one or more regions of the hydrogel scaffold may be illuminated, thereby deprotecting substrate(s) within those regions. Preferably, the glass slide containing the hydrogel may be mounted on a x-y-z motorized stage so that the location of the illuminations/deprotections may be controlled.

Incubation with Enzyme and Bioactive Signal

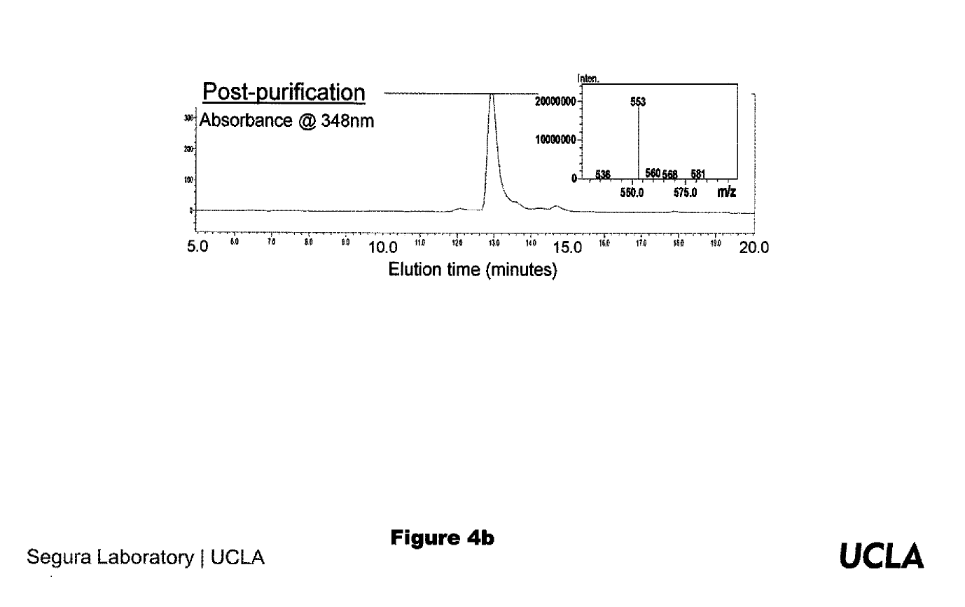

In a preferred embodiment, the hydrogel with one more deprotected substrates is incubated with the enzyme (i.e., FXIIIa) and the bioactive signal(s) (as shown in FIG. 5). Preferably, the hydrogel is incubated with a solution containing the Q-peptide-RGD and the enzyme. Preferably, to immobilize multiple bioactive signals, different regions of the hydrogel may be deprotected and reacted (i.e., incubated with the enzyme/bioactive signal mixture). For example, one site may be illuminated and thereby deprotected. Thereafter that site may be incubated with the enzyme/bioactive signal mixture. Subsequently, another site may be illuminated, deprotected, and incubated in the same fashion. In this manner, protection, deprotection, and immobilization of bioactive signal may be controlled. In other embodiments, multiple sites in the hydrogel may be illuminated, deprotected, and incubated at the same time.

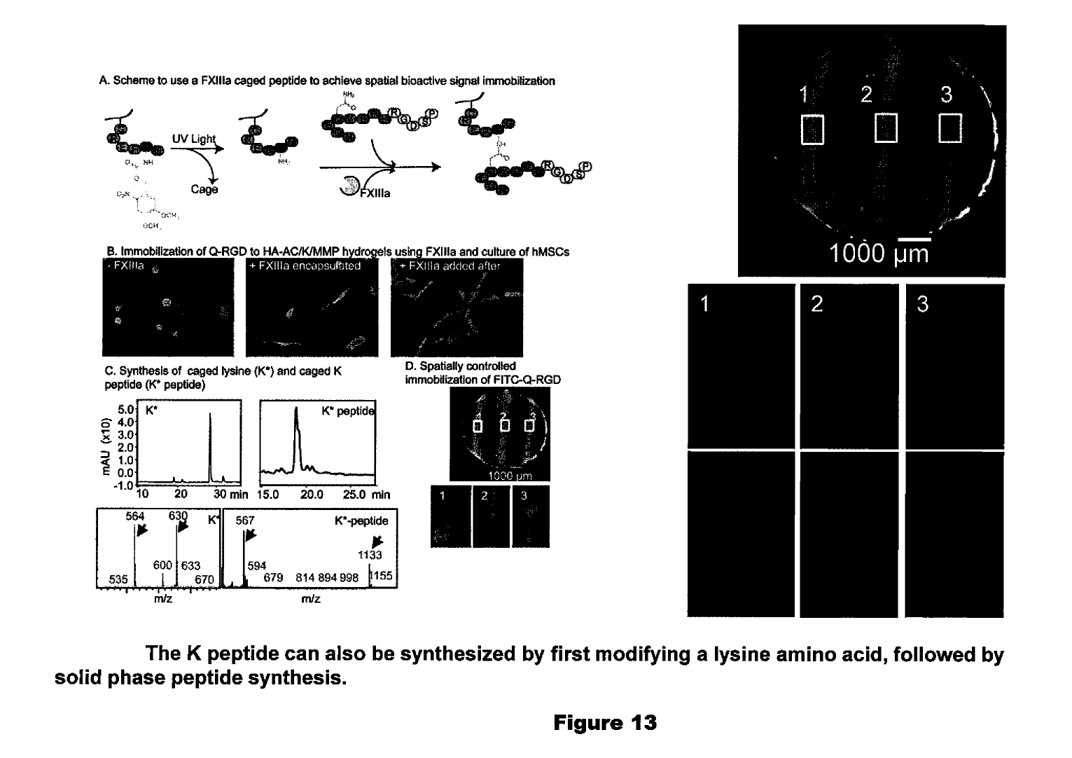

Furthermore, the methods and hydrogels described herein may be used to control cellular migration and/or introduce tunnels inside the hydrogels (as shown in FIG. 14). These methods and hydrogels will be discussed further below.

### Spacially Controlling Cell Migration

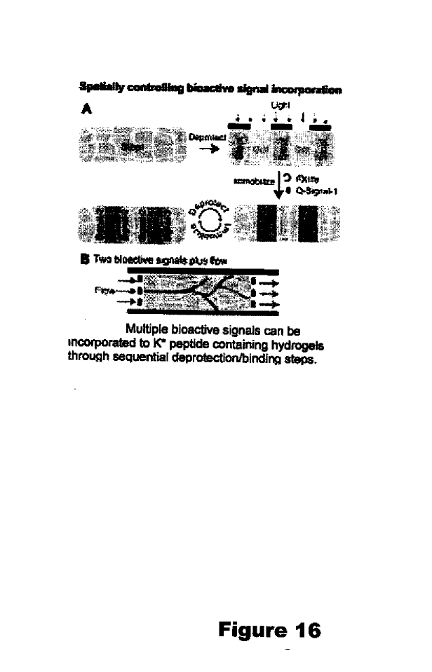

In a preferred embodiment, controlling cellular migration allows for the control of multicellular organization in three dimensions, and promotes differentiation into different cell types. Although different cell types express different proteases and the protease expression profile changes as cells differentiate and morphogenesis occurs, the differentiation of stem cells or the organization of multicellular structures have not been guided using differences in matrix proteolytic degradation within biomaterials. For example, endothelial cells have been shown to specifically degrade MMP-2 labile peptides and plasmin degradable peptides but not MMP-1 labile sequences, while fibroblast can degrade both. Thus, scaffolds that contain areas that promote MMP-1 or MMP-2 mediated degradation and areas that promote degradation by multiple proteases may be synthesized (as shown in FIG. 17). For example, cells inside peptide crosslinked PEG and HA hydrogels migrate through proteolytic degradation. [(see, e.g., Raeber G P, Mayer J, Hubbell J A. Part I: A novel in-vitro system for simultaneous mechanical stimulation and time-lapse microscopy in 3D. Biomech Model Mechanobiol 2008 June; 7(3):203-214)]. Second, HUVECs have been found to express predominantly MMP-2 and MMP-9, while fibroblast have been found to express a variety of MMPs, including MMP-1, MMP-2, MMP-3 and MMP-9 [(see, e.g., Seliktar D, Zisch A H, Lutolf M P, Wrana J L, Hubbell J A. MMP-2 sensitive, VEGF-bearing bioactive hydrogels for promotion of vascular healing. J Biomed Mater Res A 2004 Mar. 15; 68(4):704-716)]. Third, active MMP-2 binds to vβ3 integrins facilitating cellular migration [(see, e.g., Brooks P C, Stromblad S, Sanders L C, von Schalscha T L, Aimes R T, Stetler-Stevenson W G, et al. Localization of matrix metalloproteinase MMP-2 to the surface of invasive cells by interaction with integrin alpha v beta 3. Cell 1996 May 31; 85(5):683-693)]. Last, MMPs aid in morphogenesis though a variety of mechanisms including, the degradation of the ECM, the activation of ligands and the exposure of cryptic sites on proteins.

In a preferred embodiment, the following hydrogel is prepared to spacially control cellular migration. Preferably, a hydrogel is cross-linked with a caged MMP degradable peptide. MMP protease cannot degrade the caged peptide. In this manner, cells cannot migrate until network degradation occurs. Preferably, upon selective UV illumination as described herein, the MMP degradable peptide is “decaged” or “unlocked.” As such, cell-released MMP protease may degrade the uncaged peptide, and cells are able to migrate through degraded path. In other embodiments, in addition to, or in lieu of, a caged MMP degradable peptide, one or more of the following may be used.

In a preferred embodiment, for example, to control HUVEC migration, a caged MMP-2 degradable peptide sequence may be used. Hydrogels may be synthesized using a mixture of two protease degradable peptide crosslinkers, one that is not caged and is MMP-1 degradable (VPMSMP) and one that is caged and is MMP-2 degradable (IPES*LRAG) [(see, e.g., Patterson J, Hubbell J A. Enhanced proteolytic degradation of molecularly engineered PEG hydrogels in response to MMP-1 and MMP-2. Biomaterials October; 31(30):7836-7845)]. Thus, the MMP-2 degradable peptide can be deprotected at specific locations; and HUVECs may only be able to migrate in the regions of deprotected MMP-2 peptide, while fibroblasts may be able to migrate throughout the hydrogel. As such, controlling the location of HUVECs in three dimensions, may allow for the control the organization of fibroblasts, as fibroblasts typically localize next to HUVECs. As described herein, the hydrogels may be synthesized inside the microfluidic device with or without cells. Additionally, RGD may be used to promote binding through integrin receptors. Preferably, no migration results in the absence of RGD inside HA hydrogels. Deprotection of the caged peptides may be achieved using a two-photon confocal microscope or a mask/UV lamp.

### Spacially Introducing Tunnels Inside Hydrogels

One main problem with synthetic hydrogel scaffolds that are implanted is their lack of cellular infiltration at sites of low protease degradation or in hydrogels that are not protease degradable (e.g. pure HA hydrogels). Further, for hydrogels that promote infiltration the hydrogel is rapidly degraded and thus long term mechanical support to the infiltrating or transplanted cells is not possible. This prevents regeneration to be complete and to fully connect adjacent injured or diseased sites. To overcome this problem, channels may be created within a hydrogel that are degraded slowly such as the HA hydrogel. After subcutaneous implantation of protease degradable HA hydrogels, cellular infiltration may be found only at the periphery of the implant. In contrast, when macro-pores are introduced to a non-protease degradable HA hydrogel (degraded only through hyaluronidases), extensive infiltration and angiogenesis may result. As such, creating tunnels that span the entire hydrogel width, may allow for the reconnection of injured tissue and promotion of regeneration. In addition, these physical tunnels may be used to promote HUVEC tube formation and multicellular HUVEC/fibroblast organization.

In a preferred embodiment, the following hydrogel is prepared to introduce tunnels inside hydrogels. Preferably, the hydrogel may be crosslinked with only caged peptide degradable by plasmin and/or trypsin (NK*V). Preferably, upon selective UV illumination as described herein, specific regions of the hydrogel may be deprotected. Preferably, trypsin and/or plasmin may be added to the hydrogel to degrade only regions previously exposed to the selective UV illumination. The same technology as shown in FIG. 7 may be used to construct channels or tunnels inside the hydrogel except that the STOP! region is not degradable by proteases. Furthermore, the inside of the tunnel may be modified with, for example, VEGF using the FXIIIa system as described herein. The same peptide that is degraded by trypsin or plasmin may also be recognized by FXIIIa [(the sequence of the K peptide is less stringent than that of the Q peptide, see, e.g., Hu B H, Messersmith P B. Rational design of transglutaminase substrate peptides for rapid enzymatic formation of hydrogels. J Am Chem Soc 2003 Nov. 26; 125(47):14298-14299)]. Thus, deprotection may result in both trypsin activity and FXIIIa activity.

### Visualization as Morphogenesis is Happening

Current limitations with hydrogel technology include the ability to visualize the entrapped cells over time and the experimentation on the absence of flow (static conditions). For handling reasons hydrogels are often produced with more than 1 mm thickness and more than 3 mm in diameter. This allows the hydrogels to be cultured in regular tissue culture plates and handled easily; however, it limits visualization since the overall hydrogel size is too large to be imaged in its entirety and introducing flow is not possible. To overcome this limitation researchers have utilized large flow chambers or microfluidic flow chambers.

In a preferred embodiment, microfluidic flow chambers are used to cast, modify, seed cells and visualize the hydrogels described herein. For example, microfluidic chambers of 0.8 mm in height may be used because that is the working distance of standard microscope 100× objectives. Preferably, a hydrogel area of 1.1 mm in diameter is used to generate 3 μL hydrogels. This may be, however, much smaller as within 3 μL of hydrogel, around 15,000 to 60,000 cells may be cultured. However, this may allow the use of regular pipettes for hydrogel loading. To add flow, standard tubing, lour lock connectors and pump to flow liquid through the hydrogel may be used. Preferably, the microfluidic device includes (1) a port(s) for hydrogel precursor loading, (2) one or more reservoirs to hold/pump media, and/or (3) a port(s) for cell seeding if cells are introduced after hydrogel modification. In addition, the devices may be placed on top of a cover glass slide for effective imaging and UV deprotection.

In a preferred embodiment, the methodology of casting/incubating the hydrogels described herein is as follows. Preferably, the following occurs: (1) cast the hydrogel with or without cells, (2) after the hydrogel hardens, PBS (˜10 min) is flown through the hydrogel, and the hydrogel is be exposed to 365 nm light either through a lamp/mask combination or through a two-photon confocal microscope, (3) PBS is flowed to remove free cage group, for FXIIIa modified gels, (4) PBS is flowed with Q-bioactive factor and FXIIIa, (5) after incubating for 10 minutes to allow the reaction to continue, PBS is flowed again to wash. To ensure the pattern is forming, a fluorescently labeled Q-bioactive signal may be used. Preferably, for hydrogels to control cellular migration only steps 1-3 may be performed. Preferably, for hydrogels with tunnels, at step 4, trypsin or plasmin may be flown through the hydrogel to degrade the desired region. Since cells are regularly exposed to trypsin, no adverse effects are expected by exposure of the cells during channel formation. Preferably, since the devise is mounted onto a cover glass slide, a standard microscope holder may be used to visualize the sample. In other embodiments, the incubation in step (5) may be less than 10 minutes or longer than 10 minutes. In yet other embodiments, some or all of the foregoing steps, in addition to other steps described herein, may be used in the patterning/migration and/or tunneling assays.

In a preferred embodiment, the methods and hydrogels described therein may include a soluble factor concentration gradient. For example, a gradient of VEGF may be studied to determine whether it further induces the differentiation of MSCs toward ECs and pericytes (in addition to the VEGF and PDGF spatial signals).

In a preferred embodiment, the cell seeding port described above is a void in the hydrogel formed by placing an obstacle during hydrogel formation that can be removed post hardening. The void may then be used to place a cluster of cells through a syringe so that cellular migration from that location may be visualized.

### Cell Seeding and Cellular Assays

In a preferred embodiment, two type of cell seeding approaches may be used. For migration studies, cells may be seeded as a cell cluster using the cell port described above. For experiments where differentiation of cells into two cell types is studied, cells may be seeded homogeneously through the hydrogel during its formation. All the chemistry may be performed with the cells present. Preferably, to ensure the cells are alive after all the modifications are complete, the LIVE/DEAD assay, TUNEL assay and/or Ki67 staining may be used to determine viability, apoptosis and proliferation, respectively. Preferably, 90% viability exists.

### Differentiation Assays

In a preferred embodiment, cellular differentiation is studied/tracked using the following methods. Preferably, cells are cultured inside the hydrogels for up to 14 days. These cells may be fixed and stained and/or collected for mRNA extraction every 3 days (or within any other time interval) to determine their differentiation state. Preferably, the endothelial markers that are used are one or more of the following: VE-Cadherin, CD31 (PECAM), ICAM-1, von Willebrand factor (vWF), and VEGFR-2. Preferably, the pericyte markers that are used are one or more of the following: PDGFR-B, Desmin, SMA, Angiopoietin-1, Thy-1. Preferably, immunofluorescent staining and quantitative PCR with specific antibodies/primers against these molecules is used. Preferably, the effect of the biomolecule concentration, the pattern size and time on differentiation may be studied. In other embodiments, other endothelial markers may be used in lieu of, or in connection with, the endothelial markers described above. In yet other embodiments, other pericyte markers may be used in lieu of, or in connection with, the pericyte markers described above.

### Visualization of Morphogenesis

In a preferred embodiment, to aid in visualization, GFP transfected hMSCs cells (obtained from the Institute for Regenerative Medicine at Scott & White) and LIVE tracker labeled HUVECs and fibroblast as previously reported [(see, e.g., Moon J J, Saik J E, Poche R A, Leslie-Barbick J E, Lee S H, Smith A A, et al. Biomimetic hydrogels with pro-angiogenic properties. Biomaterials May; 31(14):3840-3847)] may be used. In this regard, the cells may be imaged throughout the 14 days of incubation. For example, cells may be imaged one day and cultured 2 days thereafter, and then imaged again for one day. For the cells labeled with live trackers imaging may only be done for the first 3 days since the dye fades rapidly after that.

### In Vivo Angiogenesis Assay:

In a preferred embodiment, cellular and acellular scaffolds may be used to determine if the inventive scaffolds described herein may induce vascularization in vivo (ARC #2010-017-01). Preferably, animal studies may conform to the guidelines for the care and use of laboratory animals set by the Federal Animal Welfare Act as overseen by the University of California Los Angeles. Briefly, following a 7-day acclimation period, female BALB/c mice may be anesthetized with isofluorane. The dorsal region may be clipped and prepared for aseptic surgery with povidine iodine solution. A small incision may be made down the midline using curved scissors and subcutaneous pockets may be created with blunt dissection. The hydrogel scaffolds may be inserted into the pockets and the incision may be closed with surgical staples. Preferably, the hydrogel implants are collected at 3, 7, and 14 days post surgery and processed for histochemical and immunohistochemical analysis. Specifically, explanted hydrogels may be fixed in formaldehyde, embedded in paraffin and a minimum of 10 serial sections made in 5 random locations throughout the hydrogel. Preferably, sections may be stained with H&E, Masson's trichrome, Picrosirus Red, PECAM (CD31; endothelial cells), PDGFR-B (pericytes), Ki-67 (proliferation), BrdU (proliferation), and TUNEL (apoptosis). Preferably, tissues will be analyzed for Vascular Index (#/mm²) and lumen diameter (m). In other embodiments, hydrogel implants may be collected at any other time interval(s) post-surgery; and/or any other fixing and/or staining techniques known in the art may be used without departing from the scope of the present invention.

### EXAMPLES

The Examples below are merely intended as thus and by no means intended to limit the invention disclosed herein in any way.

### Example 1

### Materials and Methods

4-(4-formyl-2-methoxy-5-nitrophenoxy) butanoic acid was synthesized as previously described [(see, e.g., Griffin, D. R.; Kasko, A. M. J Am Chem Soc 2012)]. Peptides (Ac-FKGGERC-NH₂, H-NQEQVSPLRGDSPG-NH₂, FITC-NQEQVSPLRGDSPG-NH₂, Ac-GCREGPQGIWGQERCG-NH₂) were purchased from GenScript. Methanol (Fisher, 99.9%), acetic acid (Fisher, glacial), α-picoline borane complex (Sigma Aldrich, 95%), and PEG vinyl sulfone (20,000 Da, 4-arm, JenKem, Inc.) were used without purification. Acrylate-modified hyaluronic acid (HA-Ac) was synthesized as previously described [(see, e.g., Chen, T. T.; Luque, A.; Lee, S.; Anderson, S. M.; Segura, T.; Iruela-Arispe, M. L. The Journal of Cell Biology 2010, 188, 595)].

### Synthesis of Photoprotected Peptide (K Star)

4-(4-formyl-2-methoxy-5-nitrophenoxy) butanoic acid (30.3 mg, 106 μmol) was dissolved in methanol (1.2 mL) and acetic acid (120 μL), followed by addition of the K-peptide (Ac-FKGGERC-NH₂, 834 Da) (30 mg, 35.3 μmol) and a-picoline borane complex (3.8 mg, 35.3 μmol) [(see. e.g., Sato, S.; Sakamoto, T.; Miyazawa, E.; Kikugawa, Y. Tetrahedron 2004, 60, 7899)]. The reaction was allowed to proceed overnight at room temperature. The reaction was tested by Liquid chromatography-mass spectrometry (LCMS) (5-95% Acetonitrile: water, 30 minutes) for the presence of starting material (834 Da; m/z=418) and photocaged product (1104 Da; m/z=553). Purification of the sample was achieved by preparatory high-performance liquid chromatography (HPLC) (5-95% acetonitrile:water, 60 minutes), followed by lyophilization to yield a white powder (19.2 mg, 17.4 μmol, 49%). Product was tested by LCMS to confirm purity (100% photoprotected).

### Degradation Kinetics Experiment

The photoprotected peptide was dissolved to 0.1 mM in PBS (pH 7.4). For each time point 0.75 mL of the photoprotected peptide solution was placed between two glass slides with a separation of 1 mm. The solution was then exposed to UV light (λ=365 nm; I=20 mW/cm²) for a predetermined period of time (t=0.5, 1, 2, 5, 10, 15, 30 min) 0.5 mL of the exposed solution was removed and mixed with 0.5 mL of 2,4,6-Trinitrobenzene sulfonic acid (0.01% in PBS). The mixture was allowed to react at 37° C. for 2 hours before testing for absorbance at 340 nm

### Hydrogel Fabrication (3.5 Wt %)

HA-Ac was dissolved in 0.3M TEOA buffer (pH 8.2) to achieve either a 8 wt % solution. Peptide crosslinker (Ac-GCREGPQGIWGQERCG-NH₂) (0.6 mg) was dissolved in TEOA to achieve a 0.05 mg/μL solution. Factor XIII inactive (200 U/mL) was added to the PEG solution to give a final hydrogel concentration of 20 U/mL. Photoprotected peptide was dissolved in TEOA to achieve a 5 mM stock solution. The HA-Ac was combined with K star peptide to give a final hydrogel concentration of 1000 μM and incubated at 37° C. for 15 minutes. To this solution was added the peptide crosslinker (thiol:acrylate=0.4), followed by mixing and aliquoting samples (10 μL) between two glass slides (spacer=250 μm). The hydrogels were allowed to polymerize for 25 minutes at 37° C. Following gellation the hydrogels were either used immediately or placed in Tris buffer (pH 7.4) for storage and later use.

### Enzymatic Attachment Assay

Hydrogels (10 μL) containing either unprotected K peptide or K star peptide (1000 μM) were synthesized as described above. For the K star containing hydrogels there were two subsets: (1) exposed to 365 nm light (10 minutes at 4 mW/cm²) and (2) unexposed. Subsequently, a solution (20 μL) containing 1000 μM of Q-RGD (10% by mole labeled with FITC) and thrombin (1 U/mL) was added to the vial and kept at 37° C. for 20 hours. Following the incubation period, the sample was diluted with Tris-buffered saline (TB S) (170 μL) and the fluorescent signal was compared to control samples without K peptide for the unprotected K peptide. For the K star sets, the exposed and unexposed samples were compared. The fluorescence in solution directly correlates to an increase in immobilized Q-RGD within each hydrogel. The expected Q-RGD immobilization in the exposed K star hydrogel was calculated as the product of the expected fraction of uncaged K star and the concentration of Q-RGD immobilized in the K peptide hydrogels.

### Hydrogel Patterning and Imaging

Hydrogels were exposed to light (Iₑₓₚ=4 mW/cm², λₑₓₚ=365 nm, Omnicure 1000) through patterned photomasks for 10 minute periods. Following exposure, hydrogels were functionalized using a modified literature procedure [(see. e.g., Schense, J. C.; Hubbell, J. A. Bioconjugate Chemistry 1998, 10, 75)]. Briefly, hydrogels were placed into 20 μL TBS with 50 mM CaCl₂, thrombin (1 U/mL) and Q-RGD-peptide (10% by mole labeled with FITC) (50 μM) for 20 hours. The hydrogels were then placed in 1 mL of TBS (w/out CaCl₂) and the solution was exchanged every half an hour until negligible leaching of fluorescence was observed. The hydrogels were then imaged by fluorescent microscopy (Zeiss Observer.Z1).

HES-MEC lineage: hES have been derived from previously approved hESC lines WA09 and BG01 indicating the differentiation protocol is not cell line specific. The cell line WA09 (NIH Registration Number 0062) may be used. WA09 may be grown on mouse embryonic fibroblasts (MEF) to maintain the undifferentiated state and normal karotype. For experimental differentiation, WA09 may be transferred to laminen coated dishes (1 ug/cm2) and grown in MEF-conditioned media. WA09 may be passaged 2-3× to eliminate MEF from culture before differentiation. At 80-90% confluence, MEF-conditioned media may be changed to EGM2-MV and cultured for about 20 days until a uniformly epithelial morphology occurs (aka hES-MEC). The resulting epithelial cells may be of mesoderm gene expressing lineage and capable of undergoing EMT.

The following describes materials and methods relatated to the use of Fn Fragments in the methods and hydrogels of the present invention.

Engineering of Fn Variants that Display Intergrin-Specific Binding:

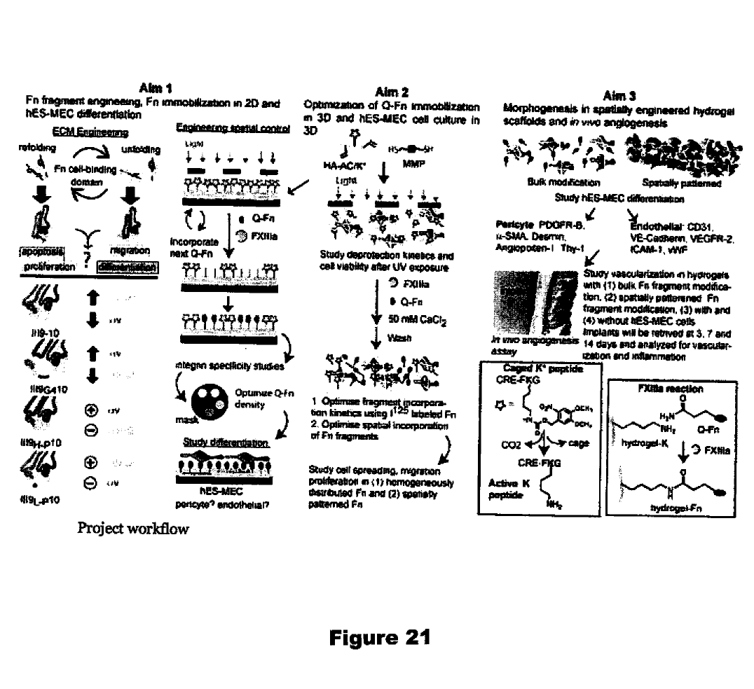

To test the role of Fn integrin-binding domain conformation on hES-MEC integrin-specific binding, the following may be done: Recombinant protein fragments of this specific region, i.e. Fn's 9^{th} and 10^{th} type III repeats (III9-10) may be generated (as shown in FIG. 21). This system relies on the fact that wild-type 1119-10 will display native binding sites for diverse integrins that can be “switched” on or off based on the conformation of the molecule. In addition to generating wild-type 1119-10, the basic amino acid sequence of the fragment may be mutated to improve inherent structural stability (III9_{L-p}10). III9_{L-P}10 may be mutated by insertion of a highly flexible polyglycine linker region (III9_{G4}10) allowing it to mimic the scenario where both domains are presented but are decoupled from each other. Finally, a loss-of-function point mutation may be inserted into the synergy site (PHRSN→PPSRN; III9_{H-P}10). Accordingly, these modified Fn fragments may allow for the presentation of different conformations of the central cell-binding domain of Fn such that the “integrin switch” is always in the “on” or “off” position and then determine the subsequent responses to the switch. The Fn fragments of the 9^{th} and 10^{th} type-III repeats displaying variable stabilities may be engineered to display a N-terminal FXIIIa crosslinking ‘handle’ to bind to the hydrogel material as well as a C-terminal Cys (for labeling/detection). [(see, e.g., 30, 31, 32, 21, 2, 20, 7)]. Fragments may be produced and purified as previously reported [(see, e.g., 7)].

### Cellular Characterization of Integrin Specificity for the Engineered Fn Variants

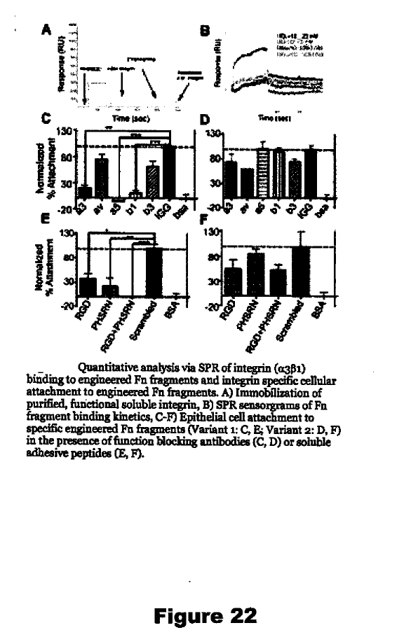

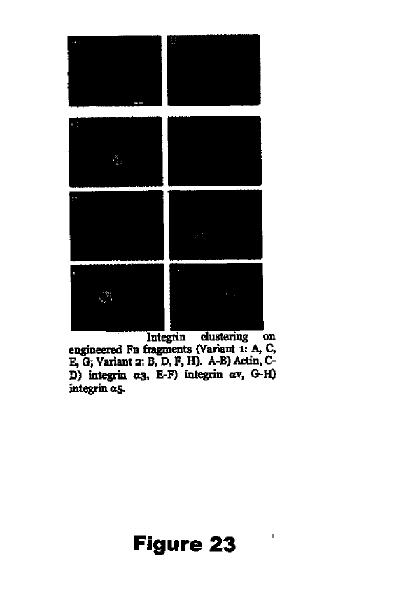

To study the integrin specificity of the engineered Fn fragments, soluble recombinant Fc-α3β1, Fc-α5β1, and Fc-αvβ3 integrins (R&D systems) may be used along with Surface Plasmon Resonance (SPR) and cell attachment assays (as shown in FIG. 22). Traditional receptor-ligand binding assays may be performed via SPR (Biacore 2000, GE Healthcare) to monitor binding kinetics and stability of interactions of soluble α3β1, α5β1, and αvβ3 integrin to immobilized Fn fragments (or vice versa) with and without synergy and RGD peptides. Cell based binding assays may be performed with hES-MECs as well as mesenchymal stem cells (MSCs). SPR and cell attachment assays may be performed in the presence and absence of synergy and RGD peptides and integrin function-blocking antibodies. As a final determinant of integrin-specific engagement of engineered Fn fragments, integrin clustering using immunofluorescence may be analyzed (as shown in FIG. 23). In order to eliminate surface ligand density as well as substrate rigidity as potential artifacts when analyzing cell responses between different fragments, 2-D cell attachment using Fragment-tethered HA hydrogels to control the surface density of fragments may be tested.

### Immobilization of Fn Variants to HA Hydrogels

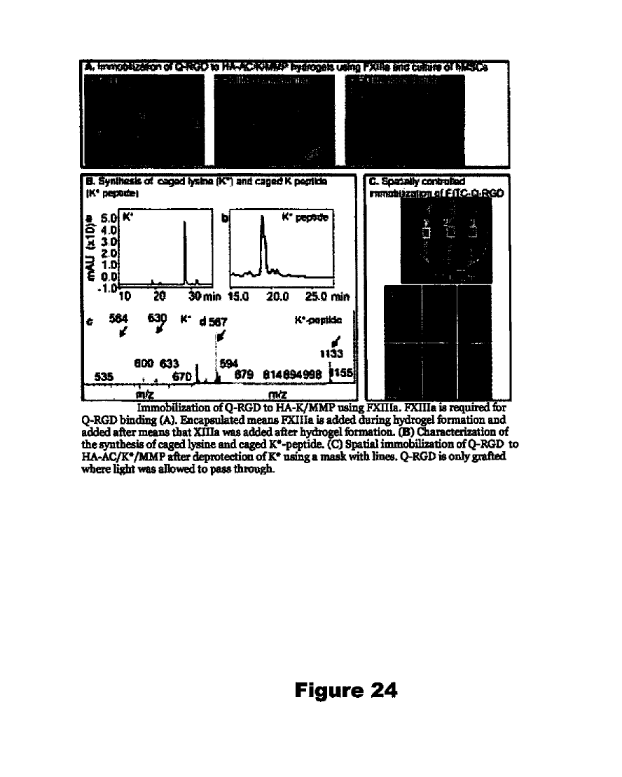

Two types of hydrogels, HAase degradable and HAase/MMP degradable, may be synthesized. Cells cultured on pure HA hydrogels do not spread and the hydrogel is negligibly degraded by 15 days of culture (longest tested) though the cells are still alive. However, when the HA is crosslinked with an MMP degradable crosslinker the hydrogels can be completely degraded by 15 days (depending on the hydrogel mechanical properties). Thus, for the studies using 2D culture, HA only hydrogels may be used, and for the experiments in 3D HA/MMP hydrogels may be used. Michael addition reactions of acrylate groups in the HA backbone (HA-AC) either HS-PEG-SH (HAase degradable) or the MMP degradable dicysteine containing peptide GCRE-GPQGIWGQ-ERCG (HAase/MMP degradable) may be used to form HA-AC/PEG or HA-AC/MMP hydrogels. As mentioned using the clot stabilization enzyme FXIIIa for bioactive signal immobilization has several advantages, including chemoselective ligation, physiological reaction conditions, and a substrate that can be caged. For example, FXIIIa may be used to decorate HA-AC/MMP hydrogels with RGD peptides after their formation, resulting in mMSC cell spreading only when FXIIIa was present (as shown in FIG. 24). Furthermore, Fn fragments synthesized with a FXIIIa substrate sequence has been shown to be crosslinked into fibrin scaffolds via FXIIIa while maintaining its adhesive activity. In these methods, the peptide FKG-GERCG (K peptide) may be introduced to HA-acrylate (HA-AC) backbone (HA-AC/K) through Michael addition prior to hydrogel formation. Subsequently FXIIIa is used to catalyze the transamination reaction between the side chain of the K in the K peptide and the side chain of the second Q in NQEQVSPL-RGDSPG (Q-RGD). This same chemistry may be used to form hydrogels and immobilize the proposed Fn fragments (Q-Fn). To immobilize the Fn fragments with spatial control, a caged lysine in the K peptide may be used. The Fmoc protected K* and the caged K* peptide may be synthesized using standard Fmoc solid phase peptide synthesis and determined that the K* peptide can be deprotected upon exposure to 365 nm UV light. In addition, grafting of Q-RGD to K* peptide modified HA-AC/MMP hydrogels results in grafting only when the HA-AC/K*/MMP hydrogel is first exposed to UV light allowing for spatially controlled immobilization.

### Bulk Immobilization of Fn Variants to the Surface of HA-AC Hydrogels:

The HA-AC/K/PEG hydrogel may be immobilized to the surface of a coverslip because it is easier to handle and allows for visualization through confocal microscopy. To immobilize the hydrogel, the surface of glass coverslips (12 mm or 25 mm) are first modified with 3-mercaptopropyl triethoxy silane to introduce thiol functional groups to the surface. Thereafter, 10 μL/12 mm of glass disk is then incubated with 4% HA-AC/K/PEG solution with a second cover glass on the surface for 20 minutes. The unmodified coverslip may be then removed leaving behind the HA-AC/K/MMP modified surface. Using confocal microscopy, the immobilized hydrogel has an estimated thickness of 150 to 200 μm. To immobilize the Q-Fn fragments, the HA-AC/K/PEG hydrogel modified glass surfaces may be incubated with a solution containing the desired Q-Fn fragment, FXIIIa and 50 mM CaCl2. They may be incubated for different times and the surface may be washed to remove the unbound fragment. Labeled Fn fragments to determine the Fn fragment density may be used. The Fn density may be determined as a function of fragment concentration and incubation time. The concentration of FXIIIa may be kept constant at 10 U/mL.

Immobilization of Fn Variants with Spatial Control

The immobilization of the Fn fragments with spatial control may allow for the screening for the optimal density of Fn ligand to achieve the desired differentiation. HA-AC may be modified with the K* peptide using Michael addition. The resulting HA-AC/K* may then be used to form a hydrogel bound to the surface of a glass cover slip. Photolithography strategy may be used to deprotect specific regions on the surface and use masks with different light transmission to deprotect different densities of the K peptide on the surface. The deprotected surfaces may then be incubated with the desired Fn variant and activated FXIIIa for the optimal time. The Fn variant modified surfaces may then be washed to remove unbound Fn variant and exposed again to long wave UV light using a different mask to deprotect an adjacent region. The same immobilization strategy may be followed. Thus, these strategies may generate two component patterns or one pattern with different densities (gradient). Thereafter, fluorescently labeled Q-Fn fragments through the N-terminal cysteine may be used to visualize and characterize the patterns as was done for Q-RGD.

### Identifying the Optimal Density of Fn Fragments:

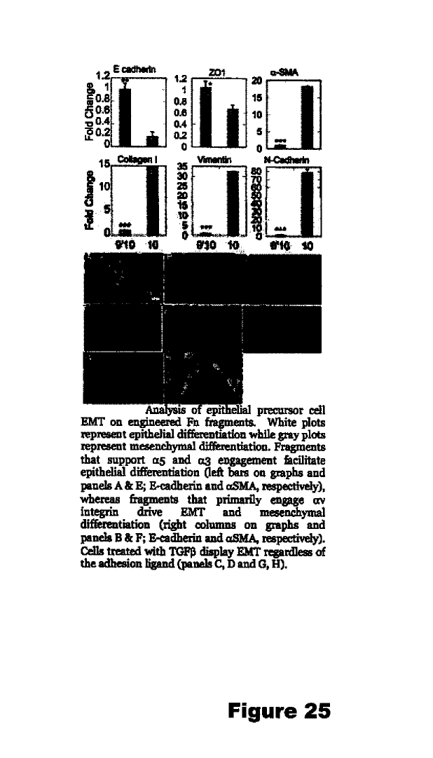

To rapidly screen which engineered Fn fragments and what density of Fn fragments achieve efficient differentiation toward pericyte-like or endothelial-like cells, multiple Fn density domains within one slide may be used. Thus rather than immobilizing the fragments at the same density over the entire surface, discrete domains with increasing fragment concentration surrounded by HA-AC/K* may be created. As such, larger numbers of Fn fragment concentration on the same surface may be screened, which may speed up imaging and media exchanges. To immobilize Fn fragments with different densities in the same surface, a mask may be generated that contains circles with different light transmissions. Thus each circle will have a different density of K* peptide deprotection and thus may result in different densities of immobilized fragment. Fn fragment concentrations starting from 0 to 1000 μM using 20 μL of HA-AC/K*/PEG gel bound to a 25 mm coverslip may be tested. The glass slides may be placed on 12-well plates and 100,000 cells may be plated on the surface and the cells may be cultured in DMEM/F12, 10% FBS, 40 ng/ml VEGF and 40 ng/mL bFGF or media without VEGF and bFGF. This media formulation may be used because it is able to support long term c0-cultures of hES-MC (a mesenchymal cell derived from hES-MEC) with HUVECs. After 5, 10, and 20 days of culture the cells may be fixed and stained for endothelial specific and pericyte specific markers following standard immunocytochemistry protocols. Those regions that display the strongest staining for the desired lineages may be selected to conduct further phenotypic analysis. The engineered Fn fragments may be capable of directing the differentiation of epithelial precursor cells toward both terminally differentiated epithelial cells and mesenchymal cell by controlling EMT (as shown in FIG. 25). These fragments may be used to drive the differentiation of hES-MEC to endothelial cells (an epithelial phenotype) and pericytes (a mesenchymal phenotype).

The endothelial markers that may be used are VE-Cadherin, CD31 (PECAM), ICAM-1, von Willebrand factor (vWF), and/or VEGFR-2. Pericyte markers that may be used are PDGFR-B, Desmin, αSMA, Angiopoietin-1, Thy-1. Both immunofluorescent staining and quantitative immunoblotting (traditional western blot and/or Bioplex analysis) may be used with specific antibodies against these molecules. In addition, the surfaces may be stained with Ki-67 (proliferation), BrdU (proliferation), and/or TUNEL (apoptosis). In other embodiments, other markers may be used.

Characterization of hES-MEC Differentiation on Fn Variant Surfaces

Using the Fn fragment type and density that promoted the most differentiation towards pericyte- or endothelial-like cells, surfaces may be generated with only one density of the Fn fragment. Because there may be more cells per surface, the cell phenotype may be analyzed using microarrays. Real time quantitative-PCR, western blotting, and Bioplex protein analysis may be used to validate the microarray findings. The microarray from surfaces that promote endothelial-like cell differentiation may show statistically significant differences from surfaces that promote pericyte-like differentiation. The microarray may be run at the UCLA microarray core, which is a per fee user facility. The microarray core also has dedicated biostatisticians, which may aid in data analysis. The hES-MEC microarrays may be compared to microarrays of HUVECs and human smooth muscle cells.

### Optimization of the Immobilization of Bioactive Signals in 3D Using a Caged FXIIIa Substrate

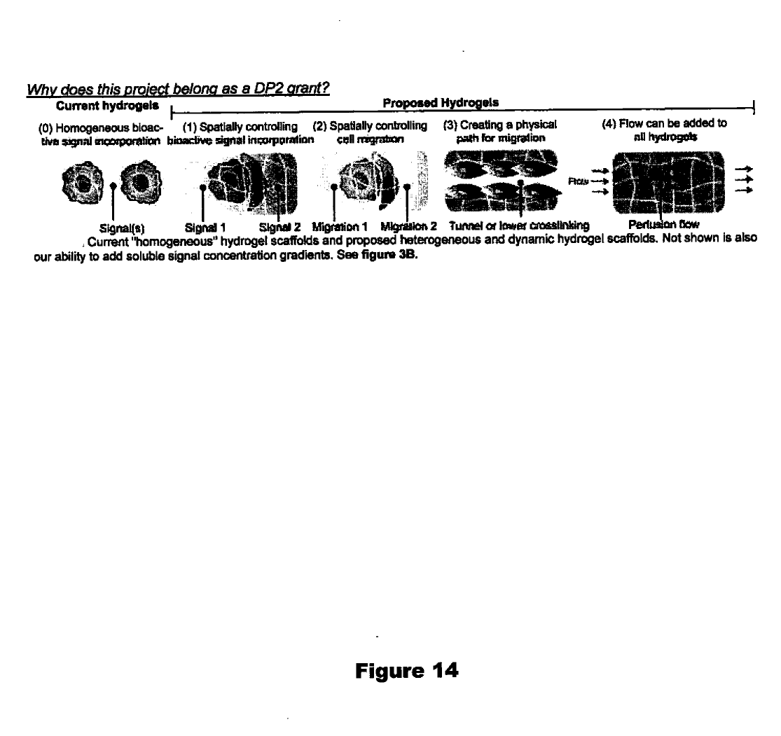

Data for the modification of HA-AC/K hydrogels with the peptide NQEQVSPL-RGDSPG shows that FXIIIa, a 200 kDa enzyme, can diffuse inside the hydrogel scaffold for a 3.5% HA hydrogel. Encapsulation of the enzyme during hydrogel formation results in the same amount or less hMSC spreading than when the enzyme was added to the Q-RGD solution post hydrogel formation, indicating that enough enzyme can diffuse inside the hydrogel and catalyze the transamination between the K and Q peptides (as shown in FIG. 15). Although diffusion does not appear to limit to the point of limiting the immobilization of Q-RGD, FXIIIa does have some diffusion limitations. The FXIIIa concentration that may be used (10 U/mL) has been previously used for the modification of fibrin gels with peptides, and the crosslinking and functionalization of PEG to form hydrogels. Since the concentration of the K peptide may reduce the number of acrylate groups on the HA, the final hydrogel storage and loss modules could change when compared to an unmodified hydrogel. Thus, the concentration of K peptide may be kept constant at 1000 μM to ensure that all the hydrogels have the same mechanical properties regardless of the concentration of Fn fragment incorporation for a given HA concentration. Nevertheless the mechanical properties of the hydrogels may be studied using rheometry to ensure that the hydrogels have similar storage and elastic modulus. [(see, e.g., 7, 29 38, 41, and 42)].

### Determination of the Optimal Deprotection Time

The time required to deprotect the caged FXIIIa substrate may depend on the method used for deprotection: (i) bulk or plane deprotection (hand held lamp) or (ii) point deprotection (two-photon confocal). In these experiments, the conditions required for complete deprotection of the caged K peptide may be determined. The deprotection kinetic experiments may be done in the absence and presence of cells to determine if cells affect the deprotection kinetics and if they are viable during and after the deprotection process. (i) Bulk deprotection: In this case the light intensity and the time of exposure may affect the deprotection kinetics. In order to study the time required for optimal deprotection, conditions used by others [see, e.g., 38, 43-48] may be used to form UV crosslinked cell loaded hydrogels, 365 nm UV light, 4 mW to 10 mW/cm². These conditions have been found to result in encapsulated cells with >95% viability. The light intensity may be kept constant and the time of exposure may be changed to determine the relationship between exposure time and percent deprotection. To monitor the deprotection reaction, the deprotected lysines may react with NHS-Alexa 555 and the fluorescence intensity may be measured using a Zeiss AxioObserved fluorescence microscope. The light intensity may be related to gels that contained 100% deprotected peptide. A linear relationship between exposure time and % deprotection is expected until 100% deprotection is reached and a plateau in fluorescence is expected. (ii) Point deprotection: In this case, a two photon confocal microscope may be used. The laser intensity (% laser), the objective used, and/or the number/thickness of scans may affect the deprotection kinetics. Initially, the same two-photon confocal microscope (LSM 710 Zeiss, two available at UCLA) with the same conditions used by recent reports [20× objective, 740 nm laser (3 W at 50% power), 1 μm scan intervals over 150 μm (or desired feature size), and a scan speed setting of 8] [see, e.g., 39 and 43] may be used. The deprotection rate as a function of time for a given laser intensity and objective may be measured. NHS-Alexa may be used to stain the hydrogel and measure its fluorescence intensity and plot intensity as a function of time to determine when a plateau is reached. Cell viability may be determined using the TUNEL assay and the LIVE/DEAD assay, which stain for DNA and membrane damage, respectively. Deprotection conditions that resulted in >95% viability may chosen for the remaining experiments.

### Immobilization of Fn Variants to HA Hydrogels

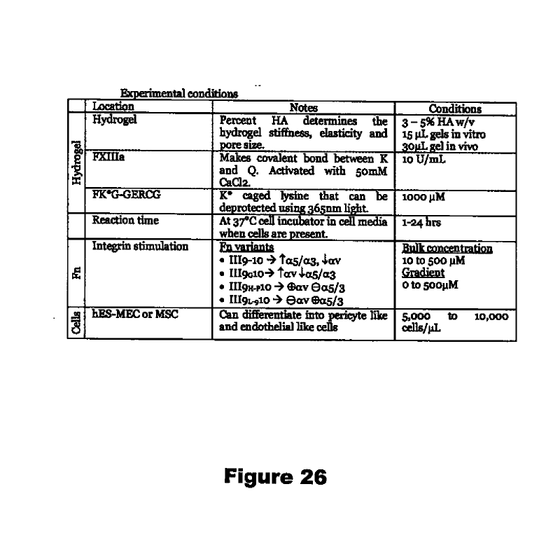

The kinetics of Fn fragment incorporation may be affected by fragment concentration, hydrogel mechanical properties, and/or time of incubation (as shown in FIG. 26). 10 U/mL of FXIIIa may be used to catalyze the immobilization reaction, the caged K* peptide may be deprotected as described herein, and the foregoing incubated in a buffer containing 50 mM CaCl2. The Fn fragments may be introduced to the CaCl2/FXIIIa solution after hydrogel formation. The amount of Fn fragment incorporated may be quantified using I¹²⁵ labeled Fn fragments and a gamma counter. Fn fragments may be labeled with I¹²⁵ using a commercial source (Perkin Elmer). The gels may be washed until no radioactivity is found in the wash and the radioactivity of negative control gels is background. The gamma counts to a standard curve may be measured to get the moles of fragment incorporated. As a negative control hydrogels that were not exposed to light may be used since they are not expected to have any fragment incorporation. As a positive control, unprotected K peptide may be used. This will be the 100% condition since all the K are available for conjugation. The fibronectin fragment concentration may be changed between 10 and 500 μM (i.e., RGD modified hydrogels may use this concentration). The pore size of the hydrogel may affect the kinetics of fragment incorporation. To determine the diffusion rate of the Fn fragments and FXIIIa into the hydrogel without reaction, protein release profiles and Fick's Law of diffusion following published protocols may be used. Diffusion of the Fn fragments may resemble that of a small molecule in water (order of 10⁻⁵ cm2/s) as was observed for proteins up to 30 kDa diffusing in PEG hydrogels. However, FXIIIa may diffuse in a slower manner. [(see, 43, 47)].

Immobilization of Two Fn Fragments with Spatial Control:

Scaffolds with two Fn fragments side by side may be synthesized. This type of hydrogel may be used to study/control the differentiation of hES-MEC into pericyte and endothelial-like cells in the same scaffold using integrin stimulation. Patterns that contain side-by-side patterns or tubes with the inner core of one fragment and the outer core of a different fragment may be created. After the immobilization of the first fragment at the desired location, the hydrogel may be washed to remove all unbound protein and deprotect the next desired location. The second fragment may then added to the 50 mM CaCl2 containing buffer/media and incubated for the optimal time. In these experiments, each fragment may be labeled with either Alexa 488 or Alexa 555 through the cysteine placed at the N-terminus of the Fn fragments for visualization. Thereafter, a fluorescence or confocal microscope may be used to visualize our three dimensional patterns.

### Cellular Characterization of the Bulk Immobilization of Fn Variants:

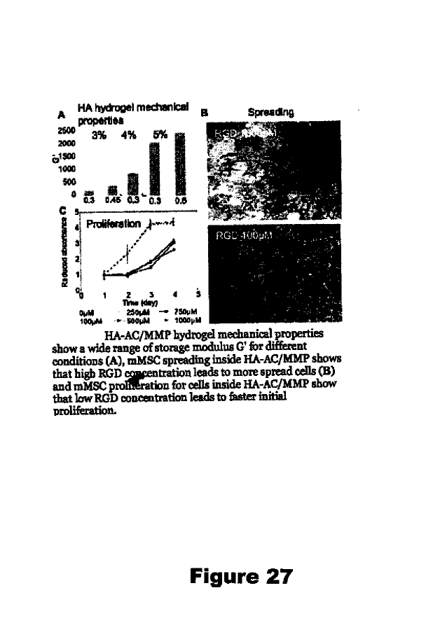

The ability of hES-MEC cells to spread, proliferate and migrate inside Fn fragment modified HA hydrogels may be important for the study of their differentiation. Hydrogels that contain one fragment homogeneously distributed may be used to determine the ideal % HA, Fn fragment concentration and cross-linking ratio to achieve spreading, migration and proliferation. Optimal spreading, migration and proliferation in a manner similar to the culture of mMSCs inside RGD modified HA-AC/MMP scaffolds is expected. For example, lower RGD concentrations may lead to higher proliferation rates, while higher RGD concentrations lead to more spread cells and no difference was found for migration (as shown in FIG. 27). hMSCs and HUVECs may be used to see how pericyte-like and endothelial cells behave in the same material. In addition, the cells may be exposed to UV light, which may be used to form the spatially patterned hydrogels to determine if cell spreading, proliferation and migration are affected.

### Cellular Viability as a Function of Cell Density

The number of cells that may be encapsulated inside our hydrogel scaffolds without loss in viability may be determined. 3000, 5000, 10,000, and 15,000 cells/μL of hydrogel may be incorporated. Immediately after hydrogel formation and 4 days thereafter, cell viability may be determined using the LIVE/DEAD assay. These methods may identify the maximum number of cells that can be encapsulated for the in vivo studies. For example, up to 15,000 iPS-neuro progenitor cells/μL of hydrogel with more than 90% cell viability.

The use of two-photon microscopy to deprotect the K* peptide should not affect cellular viability since it is very localized and low energy. However, the use of bulk or plane deprotection using a hand held UV light might affect viability of encapsulated cells. If this is indeed the case (as determined, for example, by the TUNEL assay—which assays DNA damage), the intensity of the 365 nm light used may be lowered. If the grafting is not efficient, the concentration of enzyme (the stock is 200 U/mL) and/or the K* peptide immobilized may be increased. If the caged K* is problematic after long term culture, a bulk deprotection may be performed.

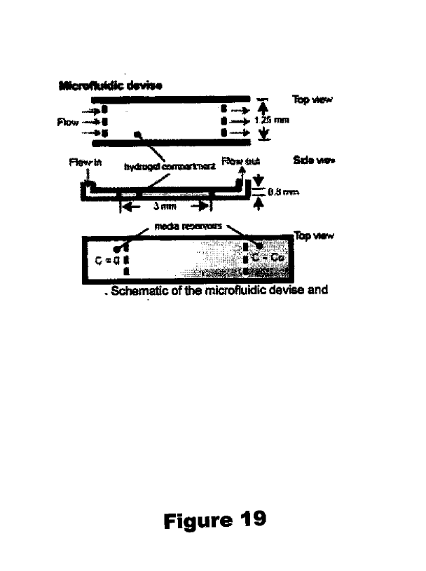

Not only is angiogenesis a critical process to normal wound repair and the realization of tissue engineering and regenerative medicine technologies, but is also critical to pathologies, such as tumor growth and fibrosis. While many tissue-engineered products have shown promising results in vitro, their translation to human use has been strikingly absent. One major limitation of these products is the extremely poor vascularization of engineered tissues, leading to oxygen depletion and eventual necrosis. Similarly, poor angiogenesis is also observed in non-healing wounds such as diabetic ulcers. In these contexts, stimulating angiogenesis is a desired goal. In contrast, over abundant angiogenic responses have been linked to progression of tumor growth and metastasis and tissue fibrosis. For these reasons, significant research efforts have been devoted to both understanding and controlling the angiogenic processes. Fundamental in vitro biomaterials knowledge may be used to test multiple variations of spatially immobilized Fn fragment decorated HA hydrogels displaying differing concentrations of the integrin specific ligand and different patterning. The differentiation of hES-MECs in 3D using a microfluidic device modified to allow real-time microscopic analysis of cellular and multicellular structures within the hydrogel in the presence or absence of an external angiogenic stimulus may be used (as shown in FIG. 19). The microfluidic in vitro model, despite the higher throughput nature, may not accurately model adult tissue responses to injury and therefore, another well-tested and accepted model for angiogenesis, a subcutaneous implant, may be employed as discussed in the examples below.

the Differentiation of hES-MEC Cultured in HA-AC/Fn Hydrogel Scaffolds

Since mechanical forces may play a role in stem cell differentiation, the experiments described below may be performed in hydrogels with storage modulus of 500 and 800 Pa. This storage modulus is based on the mechanical properties of vascularized tumors and other reports that demonstrate differentiation of MSCs toward pericyte-like or endothelial-like phenotype inside hydrogel scaffolds. The proposed hydrogel scaffolds may be synthesized to have a wide range of mechanical properties. Further, these experiments may be performed inside a microfluidic device using hydrogels, which are bulk modified or spatially modified with the engineered Fn fragment. The use of a microfluidic devise has several advantages (1) it uses less material than what we would need to use if standard tissue culture conditions would be used (7 μL hydrogel versus 30 μL), (2) since the devise is mounted on a glass coverslip photochemistry can be performed after the gel is casted inside the device, (3) since the hydrogel is only 120 μm thick imaging using confocal microscopy can be readily performed, (4) multiple devices can be mounted onto a single slide, which allows for higher throughput screens, and (5) the device allows for the introduction of soluble signals either in a gradient or throughout the hydrogel scaffold, which is ideal to determine their role in hES-MEC differentiation. [see, e.g., 35, 36, 48, and 50)].

hES-MEC Differentiation in Bulk HA-AC/Fn Hydrogels

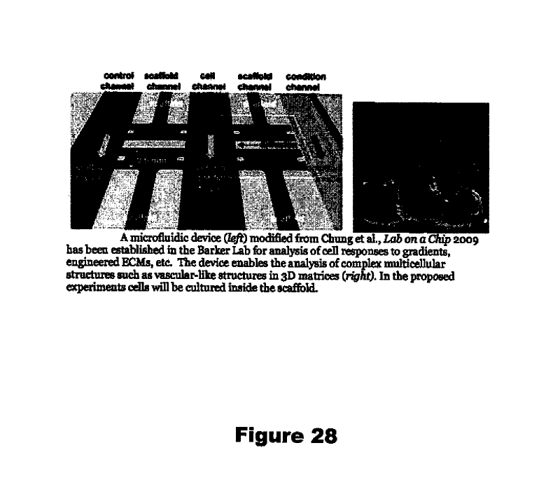

The ability of Fn fragment modified HA hydrogels to differentiate hES-MEC cells into endothelial and pericyte-like phenotypes may be studied using a microfluidic device (as shown in FIG. 28). The identified fragments that lead to hES-MEC differentiation in 2D may be used first. The fragment concentration that leads to cell spreading, proliferation and migration in 3D may be used first. This protocol is adapted from previous publications. Briefly, PDMS systems may be generated from photolithography-patterned wafers. The PDMS systems and glass coverslips may then be autoclaved, dried at 80° C. overnight, and plasma treated Immediately after plasma treatment, the glass and PDMS systems may be bonded together. The microfluidic channels may then be treated with poly-D-lysine (1 mg/ml; PDL) for 4-6 hrs at 37° C. After rinsing the channels with sterile water, the devices may be dried at 80° C. overnight Immediately after mixing the hydrogel components (HA-AC/Fn and dithiol containing peptide) and the hES-MEC, they will be introduced into the gel regions of the device and allowed to polymerize for 15 min at 37° C. Cell culture medium (DMEM/F12, 10% FBS, 40 ng/ml VEGF and 45 ng/mL bFGF or media without VEGF or bFGF) may be introduced to the microfluidic channels. The channel may be kept under static flow conditions for the duration of the experiment by maintaining a ˜40 μl droplet at the channel inlets. To exchange the medium, the droplets may be replaced daily. Cell spreading may be monitored with phase-contrast microscopy. At the final time point, cells will be fixed with 4% paraformaldehyde for 15 min, permeabilized with 0.1% Triton X-100 for 5 min, and then stained for endothelial and pericyte markers as described herein. [(see, e.g., 51)].

hES-MEC Differentiation in Spatially Patterned HA-AC/Fn Hydrogels:

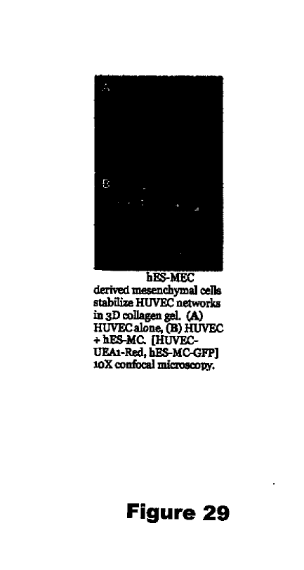

In a preferred embodiment, when hES-MECs are cultured on patterned hydrogels with environments to promote pericyte and endothelial differentiation, both phenotypes are observed. Preferably, Fn fragments may be immobilized with spatial control using the methods and hydrogels disclosed herein. Preferably, differentiation of hES-MEC into both pericyte and endothelial-like cell types based on fragment patterning which leads to more stable vessel-like networks compared to endothelial-like differentiation alone. Preferably, mesenchymal cells derived from hES-MEC are capable of HUVEC network stabilization (as shown in FIG. 29). For example, hES-MEC may be differentiated into a pericyte-like phenotype and co-cultured with HUVECs inside collagen hydrogels in DMEM/F12, 10% FBS, 40 ng/ml VEGF and 40 ng/mL bFGF media. In the absence of the pericyte-like cells HUVEC regressed (as shown in FIG. 29), while in the presence of pericyte-like cells HUVECs formed persistent 3D networks (as shown in FIG. 29).

### In Vivo Angiogenesis Assay

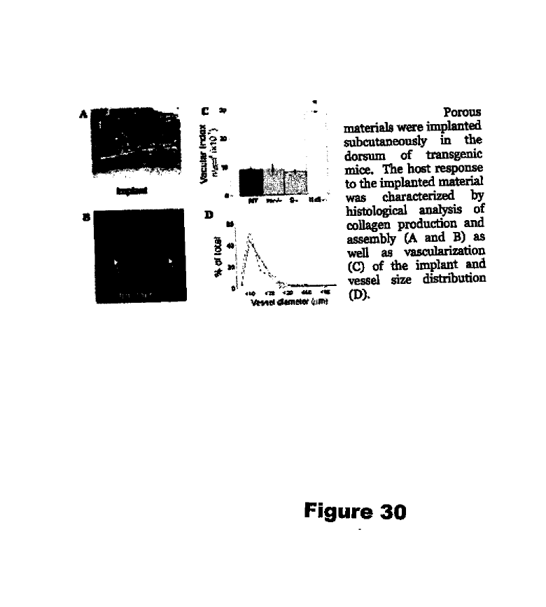

In a preferred embodiment, cellular and acellular scaffolds are used to test if HA-AC/MMP/Fn scaffolds may induce vascularization in vivo (ARC #2010-017-01). Acellular scaffolds may be used to determine the added benefit of having hES-MEC cells. Preferably, animal surgeries will conform to the guidelines for the care and use of laboratory animals set by the Federal Animal Welfare Act as overseen by the University of California Los Angeles and the Georgia Tech Institutional Animal Care and Use Committee (IACUC). Briefly, following a 7-day acclimation period, female BALB/c mice may be anesthetized with isofluorane. The dorsal region may be clipped and prepared for aseptic surgery with povidine iodine solution. A small incision may be made down the midline using curved scissors and subcutaneous pockets will be created with blunt dissection. The hydrogel scaffolds may be inserted into the pockets and the incision will be closed with surgical staples. Preferably, the hydrogel implants are collected at 3, 7, and 14 days post surgery and processed for histochemical and immunohistochemical analysis. Specifically, explanted hydrogels may be fixed in formaldehyde, embedded in paraffin and a minimum of 10 serial sections made in 5 random locations throughout the hydrogel. Sections may be stained with H&E, Masson's trichrome, Picrosirus Red, PECAM (CD31; endothelial cells), PDGFR-B (pericytes), Ki-67 (proliferation), BrdU (proliferation), and TUNEL (apoptosis). Tissues may be analyzed for Vascular Index (#/mm²) and lumen diameter (μm) (as shown in FIG. 30). In other embodiments, hydrogel implants may be collected at any other time interval, and/or any other fixation and/or staining techniques may be used without departing from the scope of the present invention.

### Acellular HA-AC/MMP/Fn Hydrogels

In a preferred embodiment, hydrogels may be synthesized as described herein. Preferably, the Fn fragments may be immobilized resembling a vascular network with the Fn fragment identified that differentiated hES-MEC into endothelial cells in the inner lumen of the pattern, and the Fn fragment identified that differentiated hES-MEC into pericytes into the outer wall of the pattern. Preferably, scaffolds with only one of the fragments may be used. Preferably, these hydrogels may be subcutaneously implanted in the back of mice as described above.

hES-MEC Transplantation in HA-AC/MMP/Fn Hydrogels

These methods may be performed in an analogous manner as described above except that cells may be encapsulated inside the hydrogel prior to implantation. To follow the most clinical relevant model for cell transplantation, media may not be introduced into these scaffolds. Cryo vials may be thawed, the number of viable cells may be counted, and the cells may be centrifuged and resuspended in PBS buffer. Preferably, the resuspended cells are entrapped within the hydrogel, and the hydrogel may be patterned. Preferably, the construct may be implanted as described above. Different cell concentrations may be tested, 3000 to 15,000 hES-MEC/μL of hydrogel, to determine the best concentration to enhance vascularization in vivo.

In a preferred embodiment, the methods described above allow for: (i) the spatial patterning of the hydrogel scaffold inside the microfluidic device, (ii) the identification of the optimal Fn fragment identity and concentration to direct differentiation toward endothelial-like and pericyte-like cells, (iii) the determination that spatially patterned hydrogels can result in the differentiation of hES-MECs into both endothelial-like and pericyte-like cells is tested and (iv) the ability of HA-AC/Fn-hES-MEC constructs to enhance vascularization in vivo is studied. Although it has been determined that HA-AC/MMP hydrogel scaffolds only swell 1.3 times their original size, this may pose difficulties when placing the hydrogel inside the microfluidic devise. In this regard, the amount of hydrogel added to the devise (5 μL for example) may be reduced and/or the amount of crosslinker may be increased. However, the use of a smaller volume means that the hES-MECs cells need to be resuspended in a smaller volume before incorporating them inside the hydrogel. Since in vitro 5000 cells/μL may be used, increasing the concentration to 7000 cell/μL is not expected to decrease viability. If static culture conditions in the microfluidic device are not sufficient to generate the desired Fn patterns (washing) or to provide nutrients for the cells, flow may be applied to the chamber to ensure proper washing and diffusion of the nutrients into the entire hydrogel.

### Vertebrate Animals

The protocol has been approved by UCLA's OARO (ARC #2010-017-01).

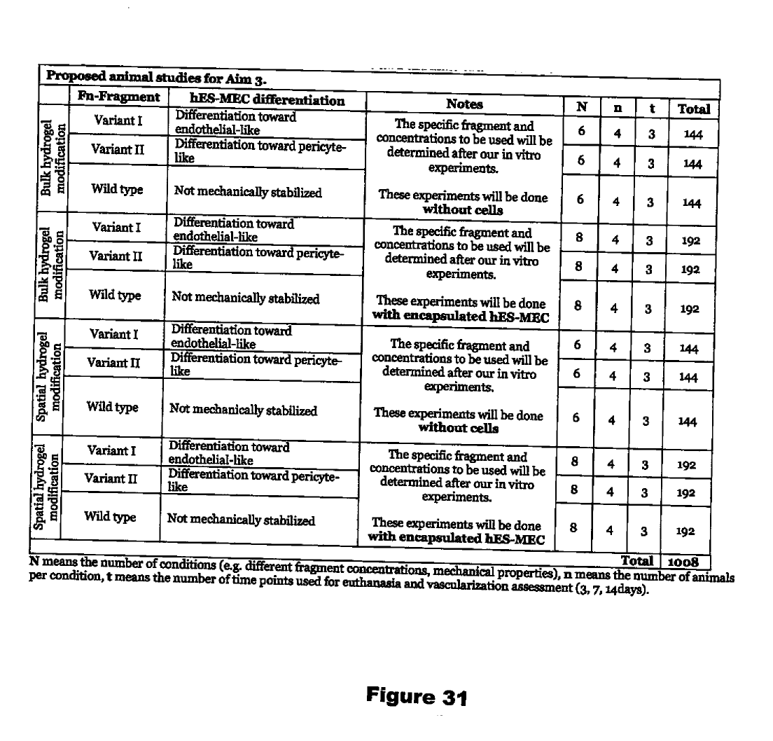

In a preferred embodiment, vertebrate animals may be used (as shown in FIG. 31). A subcutaneous model may be used along with 6-8-week old male Balb/c. Both the subcutaneous assay and Balb/c mice have been used to assess vascularization of constructs in vivo. In general, the starting reagents may be sterilized through filtering with a 0.22 μm filter. Animals may be individually anesthetized by inhaling 5% isofluorane. Once the animal no longer responds to stimuli (e.g. foot pinches), the dorsal surface may be shaved with an electric clipper and wiped with 75% ethanol at the site of surgery and prepped with betadiene. Two incisions appropriate to the size of the implant may be made in the skin aside the midline of the animal using a scalpel. Two subcutaneous pockets may subsequently be created by blunt dissection using rounded-end scissors. Into each wound, a 4-mm hydrogel or saline control will be administered. After insertion of the hydrogels, the incision may be closed with wound clips. All animal manipulations may be performed with sterile technique. Post-surgery animals may be transferred to a recovery chamber atop a heating pad. Once the animal is full ambulatory, it will be returned to a new cage. All animals may be observed daily for signs of inflammation and pain. If there is any indication that pain has persisted beyond two days, a member of the veterinary staff may be consulted. In other embodiments, other methods of studying angiogenesis in vivo are used.

A 6-8-week old male Balb/c was chosen because this strain has been previously used for subcutaneous implantation and assessment of vascularization. 4 animals per condition were chosen based on similar studies performed by the Segura Laboratory and the Barker laboratory.

### REFERENCES

1. George, E. L., Georges-Labouesse, E. N., Patel-King, R. S., Rayburn, H. & Hynes, R. O. Defects in mesoderm, neural tube and vascular development in mouse embryos lacking fibronectin. Development 119, 1079-1091 (1993).
2. Astrof, S., Crowley, D. & Hynes, R. O. Multiple cardiovascular defects caused by the absence of alternatively spliced segments of fibronectin. Dev Biol 311, 11-24 (2007).
3. Milner, R. & Campbell, I. L. Developmental regulation of beta1 integrins during angiogenesis in the central nervous system. Mol Cell Neurosci 20, 616-626 (2002).
4. Kanasaki, K. et al. Integrin beta1-mediated matrix assembly and signaling are critical for the normal development and function of the kidney glomerulus. Dev Biol 313, 584-593 (2008).
5. Tanjore, H., Zeisberg, E. M., Gerami-Naini, B. & Kalluri, R. Beta1 integrin expression on endothelial cells is required for angiogenesis but not for vasculogenesis. Dev Dyn 237, 75-82 (2008).
6. Goh, K. L., Yang, J. T. & Hynes, R. O. Mesodermal defects and cranial neural crest apoptosis in alpha5 integrin-null embryos. Development 124, 4309-4319 (1997).
7. Martino, M. M. et al. Controlling integrin specificity and stem cell differentiation in 2D and 3D environments through regulation of fibronectin domain stability. Biomaterials 30, 1089-1097 (2009).
8. Lee, S. T. et al. Engineering integrin signaling for promoting embryonic stem cell self-renewal in a precisely defined niche. Biomaterials 31, 1219-1226.
9. Gerecht, S. et al. Hyaluronic acid hydrogel for controlled self-renewal and differentiation of human embryonic stem cells. Proc Natl Acad Sci USA 104, 11298-11303 (2007).
10. Shigeri, Y., Tatsu, Y. & Yumoto, N. Synthesis and application of caged peptides and proteins. Pharmacol Ther 91, 85-92 (2001).
11. Tatsu, Y. et al. Synthesis of caged peptides using caged lysine: application to the synthesis of caged AIP, a highly specific inhibitor of calmodulin-dependent protein kinase II. Bioorg Med Chem Lett 9, 1093-1096 (1999).
12. Lovett, M., Lee, K., Edwards, A. & Kaplan, D. L. Vascularization strategies for tissue engineering. Tissue Eng Part B Rev 15, 353-370 (2009).
13. Richardson, T. P., Peters, M. C., Ennett, A. B. & Mooney, D. J. Polymeric system for dual growth factor delivery. Nat Biotechnol 19, 1029-1034 (2001).
14. Ehrbar, M. et al. The role of actively released fibrin-conjugated VEGF for VEGF receptor 2 gene activation and the enhancement of angiogenesis. Biomaterials (2007).
15. Yla-Herttuala, S. & Alitalo, K. Gene transfer as a tool to induce therapeutic vascular growth. Nat Med 9, 694-701 (2003).
16. Yancopoulos, G. D. et al. Vascular-specific growth factors and blood vessel formation. Nature 407, 242-248 (2000).
17. Carson, A. E. & Barker, T. H. Emerging concepts in engineering extracellular matrix variants for directing cell phenotype. Regen Med 4, 593-600 (2009).
18. Mardon, H. J. & Grant, K. E. The role of the ninth and tenth type III domains of human fibronectin in cell adhesion. FEBS letters 340, 197-201 (1994).
19. Mould, A. P. et al. Defining the topology of integrin alpha5beta1-fibronectin interactions using inhibitory anti-alpha5 and anti-beta1 monoclonal antibodies. Evidence that the synergy sequence of fibronectin is recognized by the amino-terminal repeats of the alpha5 subunit. The Journal of biological chemistry 272, 17283-17292 (1997).
20. Altroff, H. et al. Interdomain tilt angle determines integrin-dependent function of the ninth and tenth FIII domains of human fibronectin. The Journal of biological chemistry 279, 55995-56003 (2004).
21. Grant, R. P., Spitzfaden, C., Altroff, H., Campbell, I. D. & Mardon, H. J. Structural requirements for biological activity of the ninth and tenth FIII domains of human fibronectin. The Journal of biological chemistry 272, 6159-6166 (1997).
22. Ng, S. P. et al. Designing an extracellular matrix protein with enhanced mechanical stability. Proceedings of the National Academy of Sciences of the United States of America 104, 9633-9637 (2007).
23. Boyd, N. L., Robbins, K. R., Dhara, S. K., West, F. D. & Stice, S. L. Human embryonic stem cell-derived mesoderm-like epithelium transitions to mesenchymal progenitor cells. Tissue Eng Part A 15, 1897-1907 (2009).
24. Streicher, J. & Muller, G. B. 3D modelling of gene expression patterns. Trends Biotechnol 19, 145-148 (2001).
25. Lutolf, M. P., Gilbert, P. M. & Blau, H. M. Designing materials to direct stem-cell fate. Nature 462, 433-441 (2009).
26. Ehrbar, M. et al. Biomolecular hydrogels formed and degraded via site-specific enzymatic reactions. Biomacromolecules 8, 3000-3007 (2007).
27. Hu, B. H. & Messersmith, P. B. Rational design of transglutaminase substrate peptides for rapid enzymatic formation of hydrogels. J Am Chem Soc 125, 14298-14299 (2003).
28. Zisch, A. H., Schenk, U., Schense, J. C., Sakiyama-Elbert, S. E. & Hubbell, J. A. Covalently conjugated VEGF—fibrin matrices for endothelialization. J Control Release 72, 101-113 (2001).
29. Schense, J. C. & Hubbell, J. A. Cross-linking exogenous bifunctional peptides into fibrin gels with factor XIIIa. Bioconjug Chem 10, 75-81 (1999).
30. Barker, T. H. et al. SPARC regulates extracellular matrix organization through its modulation of integrin-linked kinase activity. The Journal of biological chemistry 280, 36483-36493 (2005).
31. Barker, T. H. et al. Thy-1 regulates fibroblast focal adhesions, cytoskeletal organization and migration through modulation of p190 RhoGAP and Rho GTPase activity. Experimental cell research 295, 488-496 (2004).
32. Krammer, A., Craig, D., Thomas, W. E., Schulten, K. & Vogel, V. A structural model for force regulated integrin binding to fibronectin's RGD-synergy site. Matrix Biol 21, 139-147 (2002).
33. Lei, Y. & Segura, T. DNA delivery from matrix metalloproteinase degradable poly(ethylene glycol) hydrogels to mouse cloned mesenchymal stem cells. Biomaterials 30, 254-265 (2009).
34. Lei, Y., Ng, Q. K. & Segura, T. Two and three-dimensional gene transfer from enzymatically degradable hydrogel scaffolds. Microsc Res Tech.
35. Adelow, C., Segura, T., Hubbell, J. A. & Frey, P. The effect of enzymatically degradable poly(ethylene glycol) hydrogels on smooth muscle cell phenotype. Biomaterials 29, 314-326 (2008).
36. Zhang, G., Drinnan, C. T., Geuss, L. R. & Suggs, L. J. Vascular differentiation of bone marrow stem cells is directed by a tunable three-dimensional matrix. Acta Biomater.
37. Silva, G. V. et al. Mesenchymal stem cells differentiate into an endothelial phenotype, enhance vascular density, and improve heart function in a canine chronic ischemia model. Circulation 111, 150-156 (2005).
38. Kloxin, A. M., Kasko, A. M., Salinas, C. N. & Anseth, K. S. Photodegradable hydrogels for dynamic tuning of physical and chemical properties. Science 324, 59-63 (2009).
39. Lee, S. H., Moon, J. J. & West, J. L. Three-dimensional micropatterning of bioactive hydrogels via two-photon laser scanning photolithography for guided 3D cell migration. Biomaterials 29, 2962-2968 (2008).
40. Luo, Y. & Shoichet, M. S. A photolabile hydrogel for guided three-dimensional cell growth and migration. Nat Mater 3, 249-253 (2004).
41. Suri, S. & Schmidt, C. E. Photopatterned collagen-hyaluronic acid interpenetrating polymer network hydrogels. Acta Biomater 5, 2385-2397 (2009).
42. Ehrbar, M. et al. Biomolecular hydrogels formed and degraded via site-specific enzymatic reactions. Biomacromolecules 8, 3000-3007 (2007).
43. DeForest, C. A., Polizzotti, B. D. & Anseth, K. S. Sequential click reactions for synthesizing and patterning three-dimensional cell microenvironments. Nat Mater 8, 659-664 (2009).
44. Moon, J. J., Hahn, M. S., Kim, I., Nsiah, B. A. & West, J. L. Micropatterning of poly(ethylene glycol) diacrylate hydrogels with biomolecules to regulate and guide endothelial morphogenesis. Tissue Eng Part A 15, 579-585 (2009).
45. Halstenberg, S., Panitch, A., Rizzi, S., Hall, H. & Hubbell, J. A. Biologically engineered protein-graft-poly(ethylene glycol) hydrogels: a cell adhesive and plasmin-degradable biosynthetic material for tissue repair. Biomacromolecules 3, 710-723 (2002).
46. Khetan, S. & Burdick, J. Cellular encapsulation in 3D hydrogels for tissue engineering. J Vis Exp (2009).
47. Weber, L. M., Lopez, C. G. & Anseth, K. S. Effects of PEG hydrogel crosslinking density on protein diffusion and encapsulated islet survival and function. J Biomed Mater Res A 90, 720-729 (2009).
48. Huebsch, N. et al. Harnessing traction-mediated manipulation of the cell/matrix interface to control stem-cell fate. Nat Mater 9, 518-526.
49. Saha, K. et al. Substrate modulus directs neural stem cell behavior. Biophys J 95, 4426-4438 (2008).
50. Discher, D. E., Janmey, P. & Wang, Y. L. Tissue Cells Feel and Respond to the Stiffness of Their Substrate. Science 310, 1139-1143 (2005).
51. Vickerman, V., Blundo, J., Chung, S. & Kamm, R. Design, fabrication and implementation of a novel multi-parameter control microfluidic platform for three-dimensional cell culture and real-time imaging. Lab on a Chip 8, 1468-1477 (2008).

All publications and patent applications cited above are incorporated herein by reference in their entireties for all purposes to the same extent as if each individual publication or patent application were specifically and individually indicated to be incorporated by reference. Although the present invention has been described in some detail by way of illustration and example for purposes of clarity and understanding, it will be apparent that certain changes and modifications may be practiced within the scope of the appended claims.

1. A method of producing a hydrogel comprising a spatially-controlled, three-dimensional distribution of one or more bioactive signals compromising:
a. illuminating the hydrogel, wherein the hydrogel comprises a polymer bound to a peptide comprising a photolabile protected amino acid, wherein at least a portion of the hydrogel is illuminated to deprotect the photolabile protected amino acid, thereby converting the photolabile protected amino acid to a deprotected amino acid, wherein the deprotected amino acid is a substrate for an enzyme in at least one portion of the hydrogel;
b. contacting the hydrogel with the enzyme and a bioactive signal, wherein the enzyme can form a bond between the substrate and the bioactive signal, thereby producing a hydrogel comprising a plurality of bioactive signals occupying three dimensions of the hydrogel within at least one portion of the hydrogel subjected to illumination.

2. The method of claim 1, wherein the polymer comprises hyaluronic acid and/or poly(ethylene glycol).

3. The method of claim 1, wherein the photolabile protected amino acid is a caged amino acid selected from the group consisting of lysine (K), aspartic acid (D), glutamic acid (E), arginine (R), serine (S), tyrosine (Y), and cysteine (C).

4. (canceled)

5. The method of claim 1, wherein the bond is a covalent bond.

6. The method of claim 1, wherein the enzyme comprises a transglutaminase.

7. The method of claim 6, wherein the enzyme is Factor XIIIa.

8. The method of claim 1, wherein the bioactive signal is selected from the group consisting of an amino acid glutamine (Q) linked to an amino acid motif RGD, an amino acid glutamine (Q) linked to one or more fibronectin fragments, and fibronectin or a fragment thereof.

9. (canceled)

10. The method of claim 3, wherein the caged amino acid comprises an ortho-nitrobenzyl photoactive chemical moiety.

11. The method of claim 7, wherein the enzyme Factor XIIIa catalyzes a transamination reaction between the deprotected amino acid and the amino acid glutamine (Q) linked to the amino acid motif RGD, thereby immobilizing the bioactive signal to the hydrogel.

12. A method of producing a hydrogel, the method comprising:
a. illuminating the hydrogel, wherein the hydrogel comprises a polymer bound to a photolabile protected peptide, and wherein one or more portions of the hydrogel is illuminated to deprotect the photolabile protected peptide, thereby converting the photolabile protected peptide to a deprotected peptide, wherein the deprotected peptide is a substrate for an enzyme in one or more portions of the hydrogel.

13. The method of claim 12, wherein the deprotected peptide is degraded within one or more portions of the hydrogel subjected to illumination.

14. The method of claim 12, wherein the peptide is a protease degradable peptide and/or comprises at least one protease cleavage site.

15. The method of claim 14, wherein the peptide selected from the group consisting of a MMP degradable peptide and a peptide degradable by trypsin or plasmin.

16. The method of claim 12, wherein the enzyme is selected from the group consisting of a protease, trypsin, and plasmin.

17. The method of claim 16, wherein the enzyme is a MMP protease.

18. (canceled)

19. (canceled)

20. The method of claim 12, wherein the peptide comprises a protease cleavage site, wherein cleavage at said site releases a bioactive signal.

21. The method of claim 1 or 12, further comprising the step of seeding the hydrogel with cells.

22. The method of claim 1, wherein the bioactive signal is one or more growth factors selected from the group consisting of VEGF and PDGF.

23. (canceled)

24. (canceled)

25. A hydrogel produced by the method of claim 1.

26. A hydrogel produced by the method of claim 12.

27. A hydrogel compromising:
a. a spatially-controlled, three-dimensional distribution of one or more bioactive signals, wherein the bioactive signals are the same or different.

28. A method of controlling cellular migration and/or introducing tunnels into hydrogels, comprising producing the hydrogel of claim 1 or 12.

29. (canceled)
